(19) United States
(12) Patent Application Publication
(10) Pub. No.: US 2026/0192078 A1
(43) Pub. Date: Jul. 9, 2026

(54) SYSTEMS AND METHODS FOR PERSONALIZED TREATMENT OF CHRONIC PAIN WITH BRAIN-COMPUTER INTERFACE AND MODULAR VIBROTACTILE APPARATUS

(71) Applicants: Washington University, St. Louis, MO (US); St. Louis University, St. Louis, MO (US)

(72) Inventors: Eric Leuthardt, St. Louis, MO (US); Simon Haroutounian, St. Louis, MO (US); Jenna Gorlewicz, St. Louis, MO (US); Peter Brunner, St. Louis, MO (US); Nabi Rustamov, St. Louis, MO (US); Phillip Demarest, St. Louis, MO (US); Joshua Adams, St. Louis, MO (US)

(21) Appl. No.: 19/129,707

(22) PCT Filed: Nov. 16, 2023
(86) PCT No.: PCT/US2023/079996

§ 371 Date: May 14, 2025

Related U.S. Application Data
(60) Provisional application No. 63/384,068, filed on Nov. 16, 2022.

(51) Int. Cl. A61M 21/00

(52) U.S. Cl. CPC A61M 21/00; A61M 2205/502; A61M 2205/582; A61M 2230/10

(57) ABSTRACT

Provided herein is a system for treating chronic pain in a patient, the system including a user computing device including a display screen and a brain-computer interface (BCI) device communicatively coupled to the user computing device. The BCI device includes a processor communicatively coupled to an EEG headset and a vibrotactile device. The processor is configured to (i) prompt, through a display screen, the patient to carry out a plurality of cognitive tasks, the plurality of cognitive tasks selected to create theta activation brainwaves in the patient, (ii) monitor progress of the patient toward reaching a predetermined theta activation goal, (iii) display, through the display screen, a visual feedback via a cue, such as a goal bar, showing the progress of the patient in relation to the predetermined theta activation goal, and (iv) activate, in response to the patient reaching the predetermined theta activation goal, the vibrotactile device to generate multi-modal sensory feedback.

### CROSS-REFERENCE TO RELATED APPLICATION

The application claims the benefit of priority to U.S. Provisional Patent Application No. 63/384,068, filed Nov. 26, 2022, the content and disclosure of which is hereby incorporated by reference herein in its entirety.

### BACKGROUND

The present disclosure generally relates to systems and methods for personalized treatment of chronic pain, and more particularly, systems and methods for personalized treatment of chronic pain with a brain-computer interface (BCI) and a modular vibrotactile apparatus.

Chronic pain in patients is one of the most common and burdensome conditions on the United States healthcare system. Specifically, the prevalence of chronic pain is about 20%, and the annual costs to imperfectly treat chronic pain exceed $560 billion. Not all chronic pain is severe and constant, but 20 million Americans suffer from high-impact chronic pain, requiring longitudinal treatment that substantially impairs the functioning, ability to work, and quality of like of the people suffering from the high-impact chronic pain. Having chronic pain, especially high-impact chronic pain, increases the risk of mental health disorders like anxiety and depression, and impacts every aspect of the lives of people living with chronic pain.

Despite advancement in understanding some of the mechanisms contributing to chronic pain, current treatment options are far from optimal. Behavioral and multidisciplinary approaches to treatment have been gaining popularity due to both safely addressing psychosocial and behavioral components of pain, but the success in the past has been moderate at best. Additionally, the high costs of behavioral treatments and the lack of highly trained providers further hinder the immediate application of such approaches nationwide.

Accordingly, the majority of chronic pain patients rely on systemic pharmacotherapy to address their pain. However, the efficacy of currently available therapeutic options is limited, and only a subset of patients achieves long-term relief or functional improvement with analgesics like non-steroidal anti-inflammatory drugs (NSAIDs), opioids, muscle relaxants, or adjuvant anticonvulsant or antidepressant medications. Many chronic pain patients eventually rely on long-term opioid treatment to help alleviate their pair despite the poor ability of opioids to improve function. Further, in recent decades, there has been an overreliance on opioids for chronic pain, which has contributed to the epidemic of opioid use disorders, overdose deaths, and addictions. There have been a multitude of attempts to develop non-addictive pharmacological alternatives to opioids, but none have been successful thus far. Therefore, innovative solutions to develop alternative treatment options for pain are needed.

Further, post-traumatic neuropathic pain (PTNP), a group of neuropathic pain conditions that arise after nerve injury or trauma, is an important cause of chronic pain with very limited treatment options. PTNP is common, particularly after surgery, and approximately 5% of the 40 million surgeries performed annually in the United States result in PTNP. Despite well-defined diagnostic criteria and taxonomy, there are currently no Food and Drug Administration (FDA)-approved drugs to treat this group of conditions. Neuromodulation-based approaches such as spinal cord stimulation and deep brain stimulation have been used for certain cases of PTNP, but these interventions are invasive and can be associated with serious adverse effects such as bleeding, central nervous system (CNS) infections, and neurological deficits. Additionally, these invasive devices are expensive, require surgical implantation, and have contraindications (e.g., uncontrolled diabetes or anticoagulant therapy) that limit widespread use in chronic pain patients. Therefore, non-invasive and safe devices for pain management that could be widely applicable to chronic neuropathic pain hold substantial promise for reducing suffering while minimizing the reliance on opioid medications.

Some of the widely available technologies used to treat chronic pain and PTNP have major problems. Specifically, transcutaneous electrical nerve stimulation (TENS) is not supported by robust evidence for efficacy in PTNP. Transcranial direct current stimulation (tDCS) is a promising, non-invasive approach for managing chronic pain; however, despite almost two decades of clinical trials, in various chronic pain conditions, the optimal dose, frequency, and duration of treatment, as monotherapy or in combination, has not been established. Further, a major challenge in the chronic neuropathic pain field has been the substantial inter-patient heterogeneity in terms of factors contributing to pain. The one-size-fits-all approach has largely failed, and precision medicine approaches need to be introduced to provide solutions to a larger sector of patients.

Therefore, there is a substantial need in the treatment of chronic pain for interventions that are highly safe, do not require surgical procedures for device implantation, can be intuitively used by patients, and can be personalized to correct underlying pathological processes that contribute to pain chronification in an individual patient.

This background section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

### BRIEF DESCRIPTION

In a first aspect, a system for treating chronic pain in a patient is provided. The system includes a user computing device including a display screen, and a brain-computer interface (BCI) device communicatively coupled to the user computing device. The BCI device includes a processor communicatively coupled to an electroencephalogram (EEG) headset and a vibrotactile device. The EEG headset is configured to be coupled to the patient and monitor brainwaves of the patient, and the vibrotactile device is configured to be coupled to an anatomic region of the patient and generate multi-modal sensory feedback. The processor is configured to (i) prompt, through the display screen, the patient to carry out a plurality of cognitive tasks, the plurality of cognitive tasks selected to create theta activation brainwaves in the patient, (ii) monitor, via feedback from the EEG headset, progress of the patient toward reaching a predetermined theta activation goal, (iii) display, through the display screen, a visual feedback via a cue showing the progress of the patient in relation to the predetermined theta activation goal, and (iv) activate, in response to the patient reaching the predetermined theta activation goal, the vibrotactile device to generate the sensory feedback.

According to another particular aspect, a method for treating chronic pain in a patient includes prompting, through a display screen of a user computing device, a patient to carry out a plurality of cognitive tasks, the plurality of cognitive tasks selected to create theta activation brainwaves in the patient, and monitoring, via feedback from an EEG headset, progress of the patient toward reaching a predetermined theta activation goal. The EEG headset is configured to be attached to the patient and to monitor brainwaves of the patient, wherein the EEG headset and the user computer device are coupled to a BCI device. The method further includes displaying, through the display screen, a visual feedback via a cue showing the progress of the patient in relation to the predetermined theta activation goal, and activating, in response to the patient reaching the predetermined theta activation goal, generating multi-modal sensory feedback, wherein the vibrotactile device is configured to be coupled to an anatomic region of the patient, and wherein the vibrotactile device is coupled to the BCI device.

Another particular aspect includes at least one non-transitory computer-readable storage medium with instructions stored thereon that, in response to execution by at least one processor, cause the at least one processor to prompt, through a display screen of a user computing device, a patient to carry out a plurality of cognitive tasks, the plurality of cognitive tasks selected to create theta activation brainwaves in the patient; monitoring, via feedback from an EEG headset, progress of the patient toward reaching a predetermined theta activation goal. The EEG headset is configured to be attached to the patient and to monitor brainwaves of the patient, wherein the EEG headset and the user computer device are coupled to a BCI device; displaying, through the display screen, a visual feedback via a cue showing the progress of the patient in relation to the predetermined theta activation goal. And activating, in response to the patient reaching the predetermined theta activation goal, generating multi-modal sensory feedback, wherein the vibrotactile device is configured to be coupled to an anatomic region of the patient, and wherein the vibrotactile device is coupled to the BCI device.

Various refinements exist of the features noted in relation to the above-mentioned aspects. Further features may also be incorporated in the above-mentioned aspects as well. These refinements and additional features may exist individually or in any combination. For instance, various features discussed below in relation to any of the illustrated embodiments may be incorporated into any of the above-described aspects, alone or in any combination.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 illustrates a system for treatment of chronic pain of a patient in accordance with at least one embodiment of the present disclosure.

FIG. 2 illustrates data and signal flows through the system illustrated in FIG. 1.

FIG. 3 illustrates a block diagram of software components of a BCI computing device of the system illustrated in FIG. 1.

FIG. 4 illustrates an example configuration of components of the system illustrated in FIG. 1 in use by a patient.

FIG. 5A illustrates control and driving circuits of a vibrotactile device of the system illustrated in FIG. 1; FIG. 5B illustrates an adjustable frame of the vibrotactile device illustrated in FIG. 5A, and FIG. 5C illustrates repositionable, interchangeable vibration nodes and buffers of the vibrotactile device illustrated in FIG. 5A.

FIG. 6A illustrates a hand-specific vibrotactile device of the system illustrated in FIG. 1, and FIG. 6B illustrates a modular vibrotactile device of the system illustrated in FIG. 1 for multi-modal sensory feedback on different parts of a patient.

FIG. 7 illustrates an example configuration of a client system in accordance with an embodiment of the present disclosure.

FIG. 8 illustrates an example configuration of a server system, in accordance with an embodiment of the present disclosure.

FIG. 9 is a table showing patient demographics and medical history.

FIG. 10 includes a table that shows the hospital anxiety and depression scores (HADS) and pain catastrophizing scale (PCS) results for all patients.

FIG. 11 is a schematic showing a BCI system that includes a dry electrode electroencephalogram (EEG) headset, a laptop, a custom-made hand vibration stimulation array (HVSA) for functionally relevant sensory feedback, and a video screen for visual feedback.

FIG. 12 generally shows an overview of BCI therapy, including a treatment timeline and therapy and BCI trial.

FIG. 13a shows each patient's intervention time; FIG. 13b shows a median BCI performance across all participants across six weeks of BCI therapy, and FIG. 13c depicts Violin plots reflecting session duration across all patients each week. FIG. 13d shows exemplar power spectral density plots comparing average θ range power during rest and neurofeedback phases of BCI training for patient.

FIG. 14 includes charts and graphs showing vibrotactile BCI therapy decreases chronic pain symptoms.

FIG. 15 includes topography plots showing qualitative changes in resting power across all electrodes, as well as a differential θ modulation before and after BCI control.

FIG. 16a shows graphs illustrating the relationship between θ modulation, BCI performance, and pain relief; FIG. 16b shows the relationship between BCI performance and θ modulation across all subjects and sessions, and FIG. 16c shows the relationship between pain relief and θ modulation across all subjects and session.

FIG. 17 shows a schematic of handedness, location, and hypersensitivity type of each patient's affected area.

FIG. 18 shows power spectral density plots showing differences in frontal θ modulation during a serial subtraction mental arithmetic task.

FIG. 19 shows plots of individual patient BCI performance and pain over BCI intervention.

FIG. 20 shows plots of weekly BPI and NPSI scores over the intervention period for individual patients.

FIG. 21 shows topography plots of brain activity across patients before and after BCI intervention.

FIG. 22 shows correlation analysis of non-θ frequencies with pain relief.

FIG. 23 includes a table showing patient quantitative sensory testing results.

FIG. 24 is a table that includes patient conditioned pain modulation results.

FIG. 25 is a table showing the number of BCI trials for each patient on each session day, and across the entire intervention period.

FIG. 26 shows a table that includes patient primary and secondary outcome measures.

FIG. 27 is a table showing methods and features of the continuous BCI used in the study.

There are shown in the drawings arrangements that are presently discussed, it being understood, however, that the present embodiments are not limited to the precise arrangements and are instrumentalities shown. While multiple embodiments are disclosed, still other embodiments of the present disclosure will become apparent to those skilled in the art from the following detailed description, which shows and describes illustrative aspects of the disclosure. As will be realized, the disclosure is capable of modifications in various aspects, all without departing from the spirit and scope of the present disclosure. Accordingly, the drawings and detailed description are to be regarded as illustrative in nature and not restrictive.

### DETAILED DESCRIPTION

In various aspects, systems, and methods for chronic pain treatment, especially for post-traumatic neuropathic pain (PTNP) conditions, are needed that are highly safe, do not require surgical procedures for device implantation, can be used intuitively by patients, and personalized to correct underlying pathological processes that contribute to pain chronification in an individual patient. The systems and methods described herein fulfill this need. The PTNP condition of a patient may result from any surgery, injury, or other traumatic event where nerves of the patient have been cut, compressed, or otherwise injured. Non-limiting examples of chronic pain conditions that may be treated using the systems and methods described herein include PTNP conditions and any other chronic pain conditions that may result from, for example, surgeries, injuries, diseases, and disorders.

The disclosed system for chronic pain treatment includes a brain-computer interface (BCI) device configured to identify individual brain rhythms associated with pain relief that are optimal for each patient to control. BCI systems have emerged as a powerful, non-invasive tool for remodeling neural circuits.

In various aspects, the BCI device may be any suitable device suitable for enabling brain-initiated functionally and sensory stimulation for the alleviation of pain. For example, the BCI device may be an electroencephalographic (EEG) BCI device that enables brain-initiated functionally relevant afferent sensory stimulation in the distribution of an injured nerve, for the alleviation of pain. In some aspects, the BCI device may translate electric, magnetic, and/or metabolic brain activity into control signals of external devices that may replace, restore, enhance, supplement, or improve the natural neural output, and thereby modify an ongoing interaction between the brain and its external or internal environment.

In various aspects, the disclosed system for treating chronic pain in a patient includes a BCI device including a processor or BCI computing device communicatively coupled to an EEG headset and a vibrotactile device. The BCI-controlled vibrotactile device is driven by frontal cortical theta rhythms in the patient that reduce pain intensity and pain interference.

Synchronization between the phase of low-frequency oscillations and the amplitude of higher-frequency oscillations, i.e., phase-amplitude coupling (PAC), has become recognized as an important mechanism for neural processing. It is thought to reflect coordination between local neuronal populations and global networks in the brain. Frontal theta-gamma PAC is reduced in chronic pain studies. Gamma range is defined as 65-100 Hz, and theta range is defined as 4-8 Hz. PAC values show spatially distinct and differential pair-wise coupling between different frequency values. These findings demonstrate that there are distinct electrophysiologic phenomena that can be used to inform and personalize BCI therapy for chronic pain, as described herein.

The technical benefits of the systems and methods described herein include: providing a highly tailored, non-invasive, non-pharmacologic treatment option not currently available for chronic neuropathic pain; providing a system that can be easy used and operated from a home of a patient, enabling accessibility and scalability of chronic pain treatment to a broad population; providing a BCI that has the clinical benefit of pain being well-defined; providing a vibrotactile device including independent vibration intensity control, an adjustable frame, and discrete vibration delivery; and providing, through the defined electrophysical changes, the mechanistic foundation to further optimize and customize BCI interventions in the future. An implementation may provide a highly tailored, non-invasive, non-pharmacologic treatment option for chronic neuropathic pain. An embodiment may include home-based design that increases the accessibility and scalability for a broad population.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

Optional” or “optionally” means that the subsequently described event or circumstance may or may not occur, and that the description includes instances where the event occurs and instances where it does not.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Any publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

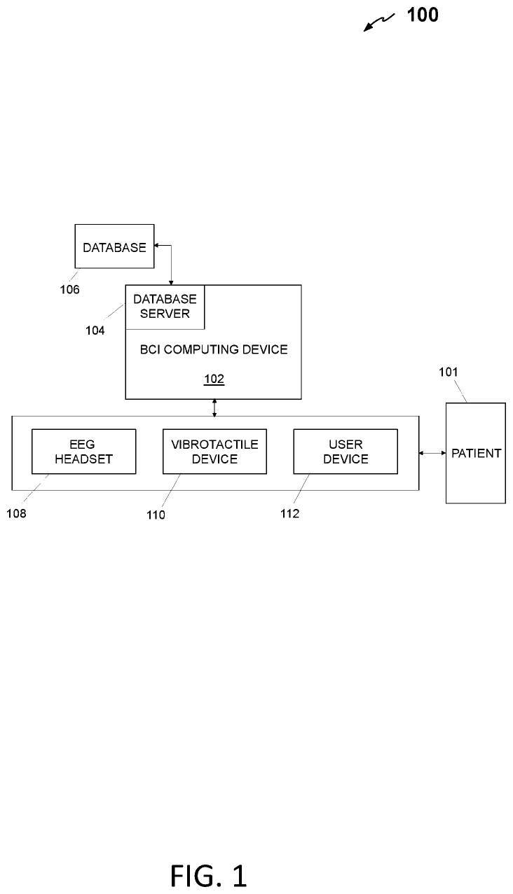

FIG. 1 illustrates a system 100 for treatment of chronic pain in a patient 101 in accordance with at least one embodiment of the present disclosure. In the exemplary embodiment, system 100 may be used for carrying out chronic pain treatment methods described herein, including for example, receiving patient feedback, controlling vibrotactile stimulation, and displaying prompts and treatment progress to patient 101.

In the exemplary embodiments, system 100 includes a brain-computer interface (BCI) computing device 102 in communication with plurality of remote devices including a database 106, an electroencephalographic (EEG) headset 108, a vibrotactile device 110, and a user device 112. In the exemplary embodiment, BCI computing device 102 receives data from the remote devices and uses that data to, for example, analyze brainwave activity of patient 101 (e.g., frontal theta activation of patient based upon data received from EEG headset 108) and progress of patient 101 toward a frontal theta activation goal (e.g., a predetermined goal specific to patient 101 stored in database 106). In the exemplary embodiment, BCI computing device 102 also sends data and other signals to the remote devices to, for example, measure and stimulate brainwave activity of patient through EEG headset 108, activate multi-modal sensory feedback and/or vibrotactile stimulation through vibrotactile device 110, and display prompts and a goal bar or other visual feedback via a cue to patient 101 through a display of user device 112. For ease of illustration, remote devices are shown in a box, and the box is shown as communicatively coupled to BCI computing device 102 and in communication with patient 101. Accordingly, it should be understood that BCI computing device 102 is individually communicatively coupled to each of EEG headset 108, vibrotactile device 110, and user device 112, and therefore, EEG headset 108, vibrotactile device 110, and user device 112 may not be communicatively coupled to each other. Further, it should be understood that each of EEG headset 108, vibrotactile device 110, and user device 112 separately interact with and/or couple to patient 101.

In the exemplary embodiment, BCI computing device 102 performs real-time data acquisition, storage, signal analysis, stimulus control, and performance tracking. Accordingly, system 100 enables patient 101 to perform brain-driven control (e.g., through EEG headset 108 in communication with BCI computing device 102) of functionally relevant vibrotactile stimulation (e.g., from vibrotactile device 110 controlled by BCI computing device) to a painful region. Further, system 100, and more specifically, BCI computing device 102 performs EEG data acquisition (e.g., on data and/or signal received from EEG headset 108), signal analysis, and stimulus control of wearable vibrotactile device 110 that delivers relevant vibrotactile stimulation to patients with chronic PTNP.

In the exemplary embodiment, EEG headset 108 includes any suitable EEG device that couples with BCI computing device 102 and vibrotactile device 110 that can be customized to an anatomy of patient 101. In the exemplary embodiment, EEG headset 108 is a dry electrode EEG headset system.

In the exemplary embodiment, vibrotactile device 110 includes any suitable vibrotactile device controlled by BCI computing device 102 that provides multi-modal sensory feedback to patient 101. In the exemplary embodiment, vibrotactile device 110 includes control and driving circuits, an adjustable frame configured to accommodate a range of anatomic regions (e.g., hands, feet, arms, legs, etc.) of patient 101, and a set of repositionable and interchangeable components configured to be removably coupled to the adjustable frame, as described further herein. Further, the set of repositionable and interchangeable components of vibrotactile device 110 include a plurality of nodes configured to (i) deliver vibration to skin of patient 101 and (ii) dampen the vibration from entering the adjustable frame, and a plurality of buffers configured to (i) further isolate the vibration and (ii) provide support at body contact points for effective fixation onto the anatomic region of patient 101. Illustrative contact points of an example may include localized vibrotactile stimulation at one to five points located on the palm, back of hand, wrist, and/or finger tips, for instance.

Each of EEG headset 108 and vibrotactile device 110 include a communication component such that EEG headset 108 and vibrotactile device 110 communicatively couple to BCI computing device 102. For example, EEG headset 108 and vibrotactile device 110 may be communicatively coupled to BCI computing device 102 through a local connection, a BLUETOOTH connection, a wireless internet connection, and/or any suitable connection that allows BCI computing device 102 to receive data from and transfer data/signals/instructions to EEG headset 108 and vibrotactile device 110.

In the exemplary embodiment, user device 112 is a computer that includes a web browser and/or a software application that enables user device 112 to access remote computer devices, such as BCI computing device 102, using the Internet or other network. More specifically, user device 112 may be communicatively coupled to the Internet through many interfaces including, but not limited to, at least one of a network, such as the Internet, a local area network (LAN), a wide area network (WAN), or an integrated services digital network (ISDN), a dial-up connection, a digital subscriber line (DSL), a cellular phone connection, and a cable modem. User device 112 may be any device capable of accessing the Internet including, but not limited to, a desktop computer, a laptop computer, a personal digital assistant (PDA), a cellular phone, a smartphone, a tablet, a phablet, wearable electronics, smart watch, or other web-based connectable equipment or mobile devices.

A database server 104 is connected to database 106. In one embodiment, centralized database 106 is stored on BCI computing device 102. In an alternative embodiment, database 106 is stored remotely from BCI computing device 102 and may be non-centralized. Database 106 may be a database configured to store information used by BCI computing device 102 including, for example, predetermined patient brainwave activity goals, historical patient data including historical progress data, and one or more vibrotactile feedback patterns for patients.

Database 106 may include a single database having separated sections or partitions, or may include multiple databases, each being separate from each other. Database 106 may store any data generated and/or received by BCI computing device 102, any patient data associated with patients 101, and any other relevant data. Furthermore, the database 106 may also store models and/or preferences of patient 101. These models and/or preferences may be set by patient 101 with user device 112.

As described herein, research has shown that changes in cortical physiology, and more specifically, theta cortical physiology, is associated with chronic pain relief. Utilizing theta-driven cortical physiology with vibrotactile devices (e.g., vibrotactile device 110) to generate (e.g., by BCI computing device 102) afferent sensory feedback that can at least temporarily relieve pain and visual feedback showing patient progress on a screen (e.g., a display device of user device 112) is an effective treatment for chronic pain alleviation.

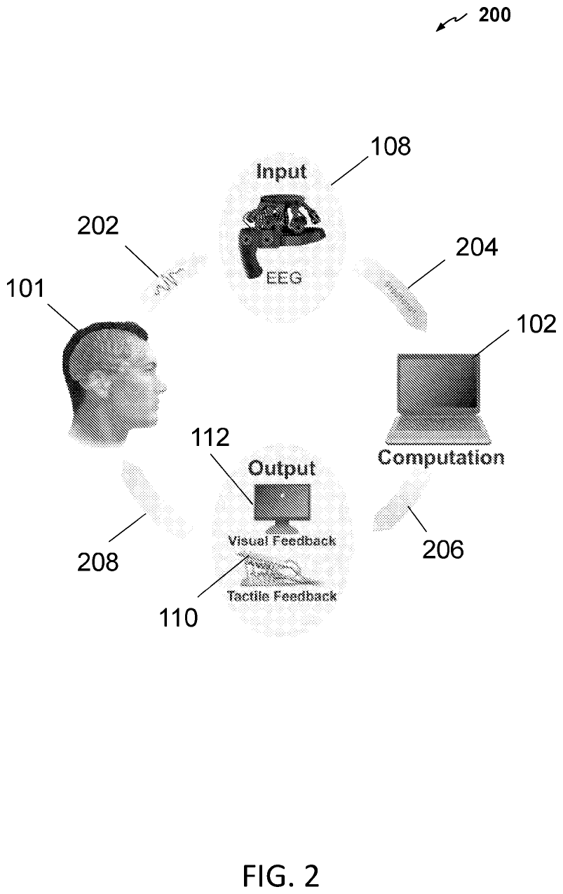

FIG. 2 illustrates data and signal flows through system 100. Specifically, FIG. 2 is a schematic of EEG-driven BCI system 100 including EEG signals as input and dual visual and vibrotactile sensory feedback.

In the exemplary embodiment, BCI computing device 102 prompts, through a display of user device 112, patient 101 to perform a battery of cognitive tasks known to create theta activations 202 in a left dorsal lateral prefrontal cortex (e.g., serial subtractions) in patient 101. Brainwaves 202, and more specifically, theta activations 202 of patient 101 are measured by EEG headset 108 coupled to a head of patient 101. Brainwave data 204 is transmitted to BCI computing device 102 from EEG headset 108 for analysis by BCI computing device 102. BCI computing device 102 compares, in real-time or near real-time, current brainwave data 204 of patient 101 with a predetermined theta activation goal specific to patient 101 (e.g., as previously determined by BCI computing device 102 and stored in database 106, shown in FIG. 1). BCI computing device 102 outputs a goal bar 206 illustrating progress of patient 101 toward the predetermined theta activation goal to be displayed on the display of user device 112. This visual feedback 208 provided to patient 101 by BCI computing device 102 through user device 112 encourages patient 101 to increase their frontal theta power to the predetermined theta activation goal. Concurrent with the increasing frontal theta power of patient 101, BCI computing device 102 also transmits signals 206 to activate and control vibrotactile device 110 such that patient 101 experiences vibrotactile stimulus 208 to an anatomical area of patient 101 affected by chronic pain when BCI computing device 102 determines that patient 101 has achieved the predetermined theta activation goal.

That is, BCI computing device 102 utilizes real-time signal processing (e.g., of brainwave data 204) to control both the movement of the goal bar displayed on user device 112 and vibrotactile feedback of vibrotactile device 110 during the task performance of patient 101. Channel frequency features of BCI computing device are specified for real-time statistical comparisons between current task performance and baseline and/or goal data. Electrodes of EEG headset 108 are selected as input channels for the weighted combination of incoming data for comparison. A frequency of 5 Hz is selected as a representative theta frequency identifier.

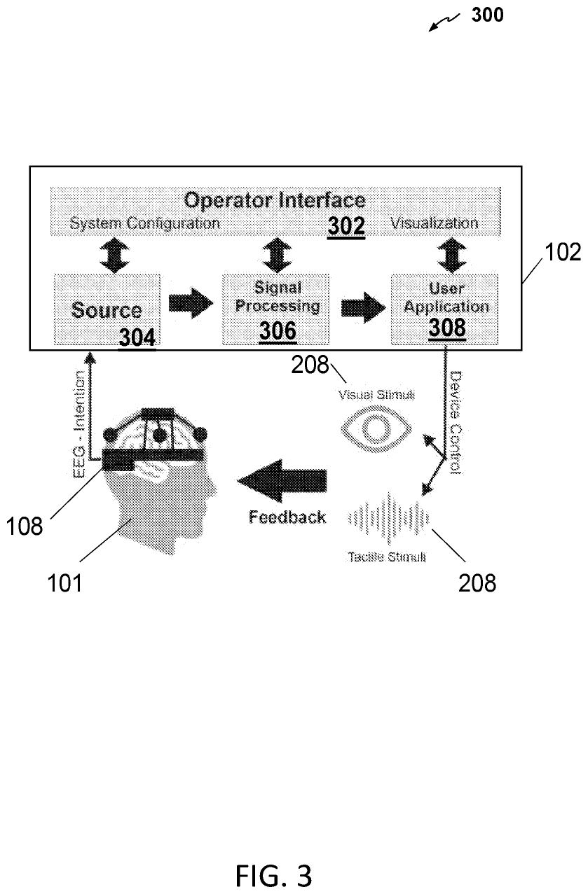

FIG. 3 illustrates a block diagram of software components of BCI computing device 102. Further, FIG. 3 illustrates how BCI computing device interacts with different components, including data/signals of system 100 (shown in FIG. 1).

In the exemplary embodiment, software architecture 300 includes four modules: an operator 302, a source 304 (e.g., for data acquisition and storage), signal processing 306, and an application 308 (e.g., for an operator interface). In the exemplary embodiment, software architecture 300 is built using an open-source modular software BCI 2000. Operator module 302 acts as a central relay for system 100 configuration and online presentation of results. During operation (e.g., in each treatment session), information is communicated from source 304 to signal processing 306, to user application 308, and back to source 304. In system 100, the feedback is provided to patient 101 in the form of both visual reward (goal bar/cursor reaching target) and vibrotactile stimuli, as described herein.

Each of modules 302, 304, 306, and 308 communicate through a documented network-capable protocol based on TCP/IP. Thus, each module 302, 304, 306, and 308 can be written in any programming language and can be run on any machine on a network. Further, because communication between modules 302, 304, 306, and 308 uses a generic protocol that can transmit all information (e.g., data, signals, instructions, variables, etc.) needed for operation of system 100, the protocol does not need to be changed when changes are made in a module 302, 304, 306, and/or 308. The information (e.g., brainwave data, stimuli instructions, etc.) that passes between modules 302, 304, 306, and 308 is standardized to minimize the dependencies between modules 302, 304, 306, and 308. Modular architecture 300 enables the BCI control and analysis methods described herein to be embedded in the architecture 300 (e.g., Pain BCI software) and be easily customizable.

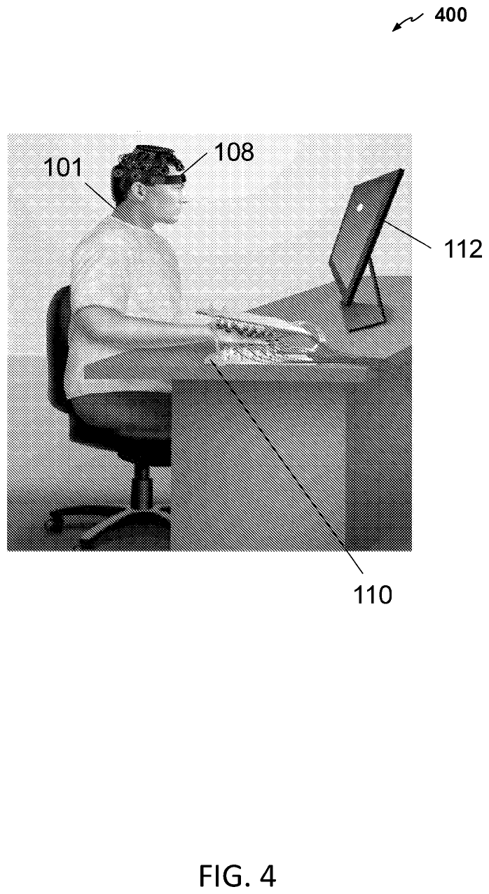

FIG. 4 illustrates an example configuration of components of system 100 (shown in FIG. 1) in use by patient 101. In the exemplary embodiment, EEG headset 108 is coupled to the head of patient 101, and a hand of patient 101 is inserted into vibrotactile device 110. Patient 101 views user device 112 for prompts and progress updates. Each of EEG headset 108, vibrotactile device 110, and user device 112 are communicatively coupled to BCI computing device 102 (shown in FIG. 1). As described herein, brainwave data from patient 101 is collected by EEG headset 108 and transmitted to BCI computing device 102. BCI computing device 102 performs data and signal analysis on the data from EEG headset 108 and determines the progress of patient 101 toward a predetermined theta activation goal. BCI computing device 102 transmits instructions for user device 112 to display the determined progress to patient 101. Further, once patient 101 meets and/or exceeds the predetermined theta activation goal, BCI computing device 102 transmits signals to vibrotactile device 110 to activate and control sensory feedback to patient 101 via vibrotactile device 110.

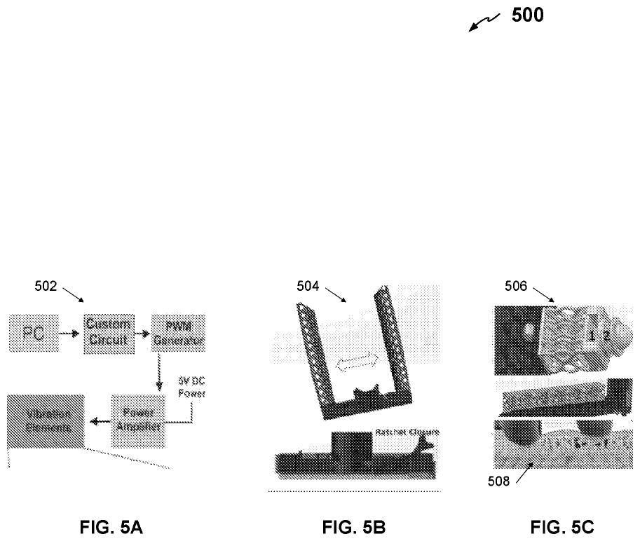

FIG. 5A illustrates control and driving circuits 502 of vibrotactile device 110. FIG. 5B illustrates an adjustable frame 504 of vibrotactile device 110, and FIG. 5C illustrates repositionable, interchangeable vibration nodes 506 and buffers 508 of vibrotactile device 110.

Control and driving circuits 502 are in communication with BCI computing device 102 (shown in FIG. 1) and enable BCI computing device 102 to control vibration output. Specifically, BCI computing device 102 sends commands to custom circuit microcontroller 502. The communication protocol between BCI computing device 102 and custom circuit board 502 is a serial port that is connected to two 24-channel Pulse Width Modulation (PWM) generators with Serial Peripheral Interface (SPI) communication protocol between them. Custom circuit microcontroller 502 decodes the commands from BCI computing device 102 and extracts 48 numbers. Custom circuit microcontroller board 502 then instructs the PWM generators (e.g., nodes 506) to generate 48 PWM signal with distinct duty cycles defined by the received numbers. The frequency of the PWM wave is 1000 Hz. Due to the high output impedance of the PWM generators, the PWM generators may not feed enough power directly to the vibration elements. A power amplifier array is introduced here to drive the elements. The working voltage of the power amplifier is 5 V (maximum total power 25 W). The vibration elements consume 0.5 W power and vibrate up to 120 Hz with 5 V of power.

Adjustable frame 504 is assembled with a mechanically retained and magnetically tensioned ratchet, and adjustable frame 504 provides a structural platform where modular vibration elements (e.g., nodes 506) and buffers 508 can attach and be easily repositioned along adjustable frame 504. Specifically, adjustable frame 504 includes (i) triangles facing interior of adjustable frame 504 for nodes 506 and buffers 508 and (ii) triangles facing outward from adjustable frame 504 for bridges to accommodate extensions for desired cutaneous distribution. Adjustable frame 504 is able to accommodate the range of size of hands, feet, and other anatomic regions of patient 101 (shown in FIG. 1) intended for treatment. The structure of adjustable frame 504 is 3D modeled (e.g., using SolidWorks), and is additively manufactured in a rigid, clear FORMLABS resin. Adjustable frame 504 includes three components: two L-shaped sides where vibration nodes 506 and buffers 508 can be fixed and a ratchet that allows easy clamping and release. The ratcheting closure and adjustable frame 504 may be tightened onto patient 101 to an appropriate pressure with one hand, which may be necessary when a caregiver requires the use of their other hand for patient 101 positioning, or if patient 101 places vibrotactile device 110 on themselves. The ratchet can be released, and vibrotactile device 110 removed, with one hand. Additionally, adjustable frame 504 provides two overarching features that are critical for fit with neuropathic pain patients with allodynia: (i) minimum points of surface area contact and (ii) no sliding or dragging motion required to place on the patients.

Nodes 506 and buffers 508 are configured to be removably coupled to adjustable frame 504. Specifically, each modular node 506 is coupled to (e.g., plugged into) adjustable frame 504 with a vibration motor slot and a pressure sensor slot. Buffers 508 are flexible and are configured to conformally deflect. All elements (e.g., nodes 506 and buffers 508) that contact patient 101 are modular and repositionable along adjustable frame 504. Nodes 506 and buffers 508 are 3D modeled and additively manufactured in a flexible FORMLABS Resin that cures to a Shore hardness of 80 A. Since nodes 506 and buffers 508 are made of flexible material, nodes 506 and buffers 508 may bend and conform to the anatomical contact point and also serve as a spring for the purpose of vibration dampening between the vibration site and the rest of the structure. This vibration dampening is unique and important, as it allows local delivery of stimuli to a defined location. In some embodiments, the dampening may range from effectiveness from around 60% and to 90%.

The elastic spring-like portion of nodes 506 and buffers 508 are multi-planar lattice structures. Alternative structure scaffolds may be implemented in alternate embodiments as well. Early tests with a Scanning Laser Doppler Vibrometer illustrate a reduction of vibration by a factor of 2+ with a vibration sink of 40 grams of mass.

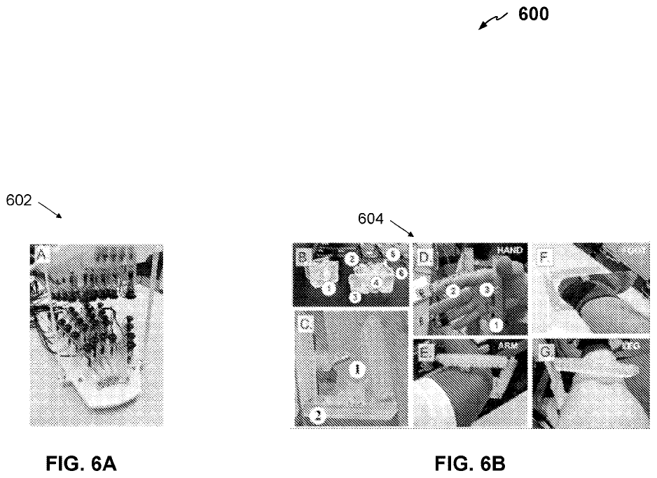

FIG. 6A illustrates a hand-specific vibrotactile device 602; and FIG. 6B illustrates a modular vibrotactile device 604 for sensory feedback on different parts of patient 101 (shown in FIG. 1). In the exemplary embodiment, both hand-specific vibrotactile device 602 and modular vibrotactile device 604 are embodiments of vibrotactile device 110 of system 100 (both shown in FIG. 1).

Hand-specific vibrotactile system 602 is used in patients with chronic distal upper extremity pain, and modular system 604 is used in other anatomical regions of patients because modular system 604 may be differentially configured to a given anatomic location. Modular system 604 is designed to be configured to a given body part (e.g., hand, foot, torso) with repositionable, vibration-isolating contact nodes. All parts are designed to be printable directly on the build platform and without support structures in stereolithographic 3D printing in 80A and rigid materials. Due to the physical sensitivities to even light friction (allodynia) of some of the target PTNP population, devices 602, 604 are designed to open and then close securely with contact nodes approaching the skin directly to minimize any tangential friction when applying or removing the device. Accordingly, devices 602, 604 create wearable vibrotactile sensory stimulating systems.

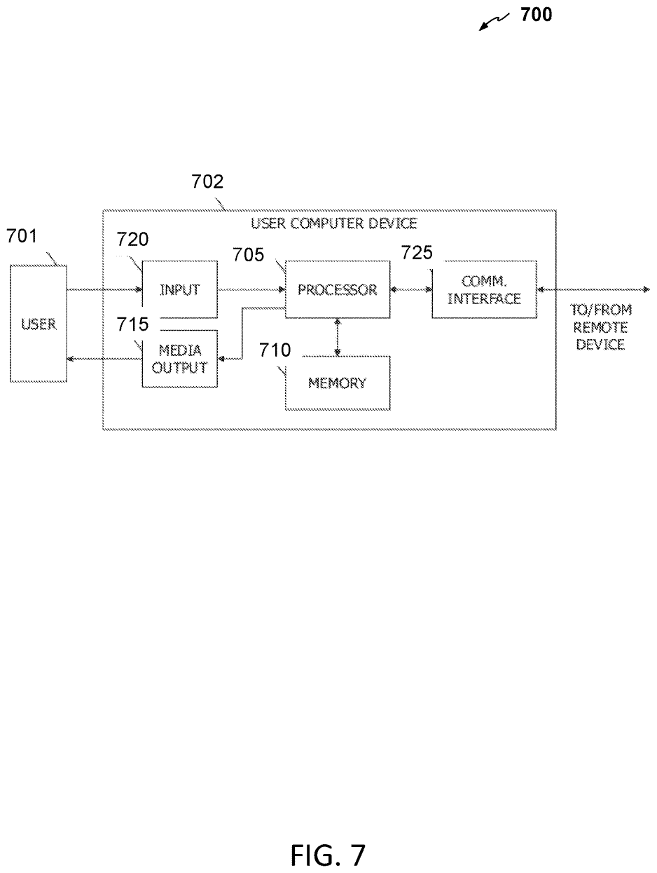

FIG. 7 illustrates an example configuration of a client system in accordance with one embodiment of the present disclosure. User computer device 702 is operated by a user 701 (e.g., patient 101, shown in FIG. 1). User computer device 702 may include, but is not limited to, user device 112 (shown in FIG. 1). User computer device 702 includes a processor 705 for executing instructions. In some embodiments, executable instructions are stored in a memory area 710. Processor 705 may include one or more processing units (e.g., in a multi-core configuration). Memory area 710 is any device allowing information such as executable instructions to be stored and retrieved. Memory area 710 may include one or more computer-readable media.

User computer device 702 also includes at least one media output component 715 for presenting information to user 701. Media output component 715 is any component capable of conveying information to user 701. In some embodiments, media output component 715 includes an output adapter (not shown) such as a video adapter and/or an audio adapter. An output adapter is operatively coupled to processor 705 and operatively couplable to an output device such as a display device (e.g., a cathode ray tube (CRT), liquid crystal display (LCD), light emitting diode (LED) display, or “electronic ink” display) or an audio output device (e.g., a speaker or headphones). In some embodiments, media output component 715 is configured to present a graphical user interface (e.g., a web browser and/or a client application) to user 701. A graphical user interface may include, for example, a goal bar of the patient, patient attributes, or the attributes of the electrical stimulation. In some embodiments, user computer device 702 includes an input device 720 for receiving input from user 701. User 701 may use input device 720 to, without limitation, apply electrical stimulation and/or vibrotactile stimulation to user 701. Input device 720 may include, for example, a keyboard, a pointing device, a mouse, a stylus, a touch sensitive panel (e.g., a touch pad or a touch screen), a gyroscope, an accelerometer, a position detector, a biometric input device, and/or an audio input device. A single component such as a touch screen may function as both an output device of media output component 715 and input device 720.

User computer device 702 may also include a communication interface 725, communicatively coupled to remote devices such as BCI computing device 102, EEG headset 108, and/or vibrotactile device 110 (all shown in FIG. 1). Communication interface 725 may include, for example, a wired or wireless network adapter and/or a wireless data transceiver for use with a mobile telecommunications network.

Stored in memory area 710 are, for example, computer-readable instructions for providing a user interface to user 701 via media output component 715 and, optionally, receiving and processing input from input device 720. The user interface may include, among other possibilities, a web browser and/or a client application. Web browsers enable users, such as user 701, to display and interact with media and other information typically embedded on a web page or a website provided by a server. A client application allows user 701 to interact with, for example, any component of system 100 (shown in FIG. 1). For example, instructions may be stored by a cloud service and the output of the execution of the instructions sent to the media output component 715.

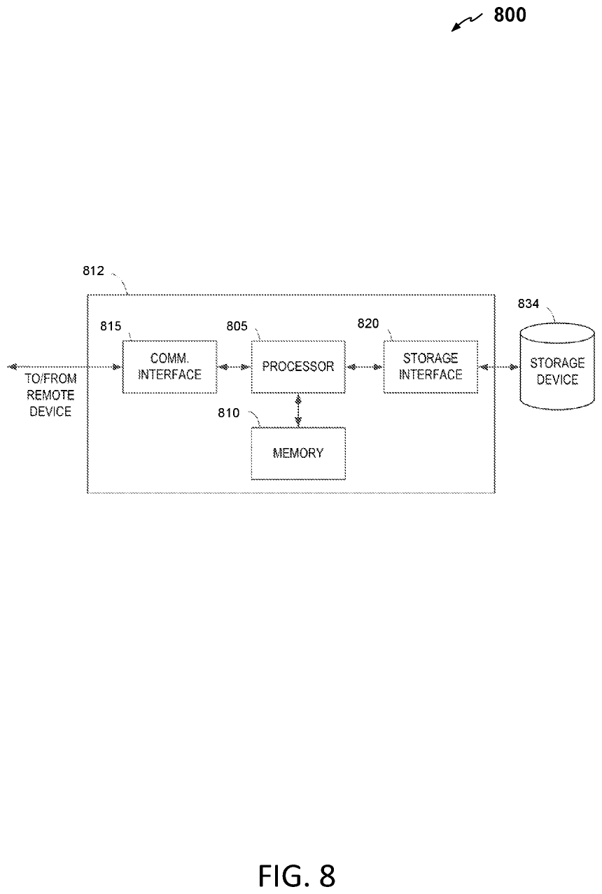

FIG. 8 illustrates an example configuration of server system 812 in accordance with an embodiment of the present disclosure. Server system 812 may include, but is not limited to, BCI computing device 102 and/or database server 104 (both shown in FIG. 1). Server system 812 includes a processor 805 for executing instructions. Instructions may be stored in a memory area 810, for example. Processor 805 may include one or more processing units (e.g., in a multi-core configuration) for executing instructions. The instructions may be executed within a variety of different operating systems on the server system 812, such as UNIX, LINUX, Microsoft Windows®, etc. It should also be appreciated that upon initiation of a computer-based method, various instructions may be executed during initialization. Some operations may be required in order to perform one or more processes described herein, while other operations may be more general and/or specific to a particular programming language (e.g., C, C#, C++, Java, or other suitable programming languages, etc.).

Processor 805 is operatively coupled to a communication interface 815 such that server system 812 is capable of communicating with remote devices such as database 106, EEG headset 108, vibrotactile device 110, user device 112 (all shown in FIG. 1), or any other suitable remote devices. For example, communication interface 815 may receive data from database 106, EEG headset 108, and/or user device 112 via wireless communication and/or the Internet.

Processor 805 may also be operatively coupled to a storage device 834, which may be used to implement database 106. Storage device 834 is any computer-operated hardware suitable for storing and/or retrieving data. In some embodiments, storage device 834 is integrated in server system 812. For example, server system 812 may include one or more hard disk drives as storage device 834. In other embodiments, storage device 834 is external to server system 812 and may be accessed by a plurality of server systems 812. For example, storage device 834 may include multiple storage units such as hard disks or solid-state disks in a redundant array of inexpensive disks (RAID) configuration. Storage device 834 may include a storage area network (SAN) and/or a network attached storage (NAS) system.

In some embodiments, processor 805 is operatively coupled to storage device 834 via a storage interface 820. Storage interface 820 is any component capable of providing processor 805 with access to storage device 834. Storage interface 820 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 605 with access to storage device 634.

Memory area 810 may include, but is not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are exemplary only and are thus not limiting as to the types of memory usable for storage of a computer program.

The methods and systems described herein may be implemented using computer programming or engineering techniques including computer software, firmware, hardware, or any combination or subset thereof, wherein the technical effects may be achieved by performing at least one of the following steps: (i) prompt, through a display screen of a user computing device, a patient to carry out a plurality of cognitive tasks, the plurality of cognitive tasks selected to create theta activation brainwaves in the patient, (ii) monitor, via feedback from an EEG headset of the patient, progress of the patient toward reaching a predetermined theta activation goal, (iii) display, through the display screen, a goal bar showing the progress of the patient in relation to the predetermined theta activation goal, and (iv) activate, in response to the patient reaching the predetermined theta activation goal, a vibrotactile device to generate sensory feedback.

In summary, early efforts in using BCI systems in pain intervention included small studies without adequate controls, had systems not customized for pain, and resulted in mixed clinical results. The majority of these previous BCI systems have used visual feedback related to brain signals associated with movement intentions. For a system to be effective in neurorehabilitation of pain circuits, visual feedback alone is insufficient. It is also essential to temporally couple relevant cortical physiology with somatosensory feedback relevant to the BCI-controlled physiology. As an example, in the setting of chronic stroke, it is critical to couple brain signals associated with ipsilateral motor intentions with proprioceptive kinematic feedback provided by a robotic exoskeleton to achieve a functional improvement. In the setting of chronic pain, it is essential to couple cortical signals associated with pain relief (e.g., increased frontal theta power) with pain alleviating vibrotactile sensory stimulation in the distribution of neuropathic pain to best enable neural remodeling to occur. Taken together, there is a need for the customized pain BCI system (e.g., system 100, shown in FIG. 1), as described herein, that (i) identifies a personalized cortical physiology associated with pain relief, (ii) enables a patient to modulate their pain relief physiology, and (iii) couples these brain signals with non-painful vibrotactile afferent sensory input to enhance central neural remodeling.

The systems and methods described herein represent a substantial departure in current methods of treating PNTP by expanding the role a BCI device can have in the rehabilitation of maladaptive neural circuits in chronic neuropathic pain. As described herein, preliminary studies demonstrate that there are pain-specific changes in brain rhythms (as measured with EEG). The changes correlate with changes in chronic pain in humans, and their targeted modulation with BCI devices result in the alleviation of chronic pain. These findings strongly suggest that cortical rhythms can be harnessed with BCI methodologies to modulate the pain-induced pathologic neural network architecture. Further, augmenting frontal theta rhythms with a BCI devices and defining its effects. Accordingly, the systems and methods described herein substantially alter strategies in treating PNTP by rehabilitating sensory perception, improving quality of life, and reducing the need for opioid therapy.

Complex regional pain syndrome (CRPS) is rare but disabling pain condition typically affecting a single extremity. The two types of CRPS are type I (previously called reflex sympathetic dystrophy) and type II (previously called causalgia). CRPS is more common in women than in men, and the incidence reaches its peak in the fifth decade. It involves one limb in most cases, and there is a history of noxious traumatic injury with or without nerve involvement. Postamputation pain (PAP) presents as a heterogeneous group of overlapping pain syndromes that occur after amputation, including phantom pains.

The systems and methods described herein may be used in the treatment of back and neck pain, including, for example, vertebral bodies, fractures, neoplastic lesions, primary metastatic, metabolic derangements, osteoporosis, Paget disease, osteitis fibrosa, hyperthyroidism, Cushing syndrome, infections, osteomyelitis, intravertebral disks, structural lesions, degeneration and herniation, chondromalacia, infections diskitis, ligaments, acute strain, involvement with other conditions, muscle structures, primary myofascial pain, secondary myofascial pain, joints, facets joints (zygapophysial or “Z” joints), osteoarthritis, mechanical arthropathy, synovial impingement, meniscoid entrapment, sacroiliac joints, mechanical arthropathy, pregnancy, degenerative and inflammatory osteoarthritis, ankylosing spondylitis, psoriatic arthritis, Reiter syndrome, chronic inflammatory bowel disease, nerve roots (and dorsal root ganglia), compressive lesions, neuroradiculopathies, meninges, arachnoiditis scar tissue, epidural space, hematoma infection (abscess), multifactorial structural, spine instability, Kyphosis Scoliosis Spondylolithiasis, degenerative spinal stenosis, spondylosis trauma infection, psychosocial, and referred pain.

The systems and methods described herein may be used in the treatment of discogenic pain, radicular pain, facet arthropathy, spinal stenosis, sacroliliac arthropathy, and headaches and head pain (e.g., primary headaches, migraines, tension-type headache, trigeminal autonomic cephalalgia, other primary headache disorders, secondary headaches, headaches attributed to trauma or injury to the head and/or neck, headache attributed to cranial or cervical vascular disorder, headache attributed to non-vascular intracranial disorder, headache attributed to a substance or withdrawal, headache attributed to infection, headache attributed to disorder or homeostasis, headache or facial pain attributed to disorder of cranium, neck, eyes, ears, nose, sinuses, teeth, mouth, or other facial/cervical structures, headache attributed to psychiatric disorders, painful cranial neuropathies, facial pains, and other headache disorders). Diskogenic pain is believed to occur as a result of three main causes, including disk infection, torsion injury, and internal disk disruption (IDD). Radicular pain originates as a result of irritation of a nerve root due to a herniated nucleus pulposus or degenerative neuroforaminal narrowing. The most commonly affected levels in the lower back are L4-L5 and L5-S1 and C5-C6 and C6-7 in the neck, although any level can be involved. For facet arthropathy, the facet, or zygapophyseal, joints are true synovial joints whose main function is to limit rotation and resist compression during lordosis. Facet joint pain results from conditions that increase the load on them, such as arthritis, decreased disk space, and increased lordosis (as in obesity). The medial branch of the dorsal ramus from the level above and the same level innervate the facet joints. These medial branches also contribute to overlapping innervation of the multifidus muscle. It is estimated that the facet joints can be implicated as the pain generator in low back pain in 15-45% of cases. Spinal stenosis results from narrowing of the central canal, lateral recesses, or neural foramina due to age-related degenerative changes, including osteophyte formation, facet hypertrophy, ligamentum flavum hypertrophy, and diffuse broad-based disk bulge. Its occurrence in the cervical spine is referred to as cervical spondylosis. Anatomical and radiographic definitions do not correlate to the presence or severity of symptoms. Stenosis in the cervical or thoracic regions can cause nerve root and/or spinal cord compression, resulting in axial, radicular, and/or myelopathic pain and sensorimotor compromise. For sacroiliac arthropathy, the sacroiliac joint connects the sacrum to the iliac bones. It is subject to degenerative and inflammatory arthritides as well as mechanical dysfunction.

The systems and methods described herein may also be used to treat musculoskeletal pain syndromes, including, for example, temporomandibular disorders (TMDs), myofascial pain syndrome (MPS), osteoarthritis, and rheumatoid arthritis. Temporomandibular disorders (TMDs) are a common set of orofacial, musculoskeletal conditions that afflict 5-12% of the US population, with costs exceeding $4 billion annually. Patients typically present with pain over the temporomandibular joint (TMJ) or muscles of mastication. This may be associated with temporal headaches, ear pain, tinnitus, clicking/popping of the TMJ, or locking of the jaw. Myofascial pain syndrome (MPS) is a common cause of soft tissue pain. MPS can occur primarily or can present as a reactive component to other conditions, such as a radiculopathy. The pathognomonic feature of MPS is the myofascial trigger point (TrP), a localized, tender, and firm or taut region within muscles or their fascia. Osteoarthritis typically involves multiple joints, and those most commonly involved are the metatarsophalangeal (MTP) joint of the great toe (hallux valgus or “bunion”), proximal interphalangeal (PIP) and distal interphalangeal (DIP) joints of the fingers, and carpometacarpal (CMC) joint of the thumb, hips, knees, and both lumbar and cervical spines. Rheumatoid arthritis (RA) is a chronic multisystem disease with inflammatory polyarthritis of symmetric distribution affecting the peripheral joints of the hands (sparing DIP joints), feet, wrists, elbows, shoulders, hips, knees, and ankles. The cervical spine is generally the only axial skeleton affected by RA.

Limitations in chronic pain therapies necessitate interventions that are effective, accessible, and safe. Brain-computer interfaces (BCIs) provide a promising modality for targeting neuropathology underlying chronic pain by converting recorded neural activity into perceivable outputs. Recent evidence suggests that increased frontal theta power (4-7 Hz) reflects pain relief from chronic and acute pain. Further studies have suggested that vibrotactile stimulation decreases pain intensity in experimental and clinical models.

A longitudinal, non-randomized, open-label pilot study was conducted with the objective of reinforcing frontal theta activity in six patients with chronic upper extremity pain using a vibrotactile neurofeedback BCI system. Patients increased their BCI performance, reflecting thought-driven control of neurofeedback, and showed a significant decrease in pain severity (1.29±0.25 MAD, p=0.03, q=0.05) and pain interference (1.79±1.10 MAD p=0.03, q=0.05) scores without any adverse events. Pain relief significantly correlated with frontal theta modulation. These findings highlight the potential of BCI-mediated cortico-sensory coupling of frontal theta with vibrotactile stimulation for alleviating chronic pain.

Conventional approaches for treating chronic pain have limited therapeutic benefits and are often associated with side effects. Only 30-40% of patients with chronic pain achieve meaningful pain relief with pharmacotherapy. Many of these patients receive opioids, which are associated with increased risks of dependence and substance use disorders. Invasive approaches for treating chronic pain, such as deep-brain stimulation and spinal cord stimulation, are effective in some patients but are expensive and can lead to complications. Given the current limitations in treating chronic pain, there is a critical need to develop safe, non-invasive, and affordable therapies.

Chronic pain is associated with pathological changes in neural circuitry, including maladaptive neuroplasticity, cortical reorganization, and changes to descending pain modulation pathways. In humans, brain-computer interface (BCI) systems have emerged as a promising tool for remodeling neural circuits. This premise of BCI relies on Hebbian learning—i.e., activity-dependent synaptic plasticity where concurrent neuronal activations strengthen neural connections. BCIs can decode cortical physiology in real-time and provide temporally precise outputs to enable the necessary conditions for Hebbian learning. This physiologic and temporal precision enables powerful conditions for enhancing neuroplastic changes. Specifically, BCIs enable robust cortico-sensory coupling, where a desired brain physiology is amplified with functionally relevant sensory feedback that is provided in real-time and with high temporal precision. This BCI-mediated method has enabled therapies for what have been considered intractable neurologic conditions, such as chronic stroke-induced hemiparesis. Early efforts in using BCI as a pain intervention have been attempted with mixed results and rarely report relationships between physiology and outcomes. This variable effect is partly due to the brain signals used and the feedback provided. Specifically, past BCIs have commonly used cortical sensorimotor rhythms associated with motor imagery and visual feedback. Neither of these inputs and outputs are directly relevant to pain perception. As described herein, in the setting of chronic pain, cortical signals associated with pain relief with pain-alleviating sensory input, such as vibrotactile stimulation may be coupled to best enable neural remodeling to occur. Further, the variation in brain regions previously reported in pain processing justifies targeted reinforcement of physiology consistently observed in multiple contexts. Particular embodiments provide relief of both chronic pain and experimental acute pain is associated with increased frontal theta (θ) rhythms (4-7 Hz).

Increases in frontal θ may occur during cognitive tasks involving attention and concentration (such as working memory tasks, mental arithmetic, and meditation). Some of these tasks have implications for pain management, such as the positive clinical outcomes associated with meditation or the phenomenon of distraction-induced analgesia. Changes in θ have observed variable results, with some reporting increases, decreases, or no changes. The locations of reported changes in θ may also be highly variable, with most studies only reporting changes in global activity. Further, known studies are primarily associative and are unable to decouple biomarkers of pathophysiology (i.e., active contributors of pathology) from biomarkers of homeostatic responses (i.e., compensatory mechanisms that reduce pathology). This limitation of previous studies prevents inferences about how a particular EEG activity contributes to symptoms of pain and only provides evidence of co-occurrence. As such, approaches that reinforce pain-related biomarkers and subsequently observe their effect on pain-related symptoms may be beneficial for interpreting the relationship between EEG features and pain symptoms.

An embodiment may include a theta-controlled vibrotactile brain-computer interface to treat chronic pain. The embodiment of the BCI system may be controlled by frontal θ rhythms and uses vibrotactile stimulation of the affected area as a means of neurofeedback in patients with medically refractory chronic upper extremity pain.

A present study aimed to test the feasibility of BCI-mediated reinforcement of frontal θ rhythms, and whether this physiology can be linked to symptom relief in chronic pain patients. Furthermore, using secondary analysis, mechanistic evidence related to BCI therapy was identified that warrants future elucidation and study design refinement. A longitudinal intervention with such a BCI-controlled vibrotactile system may be feasible, safe, and reduce pain severity and pain interference as measured by the Brief Pain Inventory (BPI). The study tested the feasibility and initial efficacy of treating chronic upper extremity pain with a BCI-controlled vibrotactile system and identified potential neurophysiological correlates of the observed clinical outcomes. Taken together, results provided compelling evidence that BCI-mediated cortico-sensory coupling may provide an approach to alleviating chronic pain in the upper extremity.

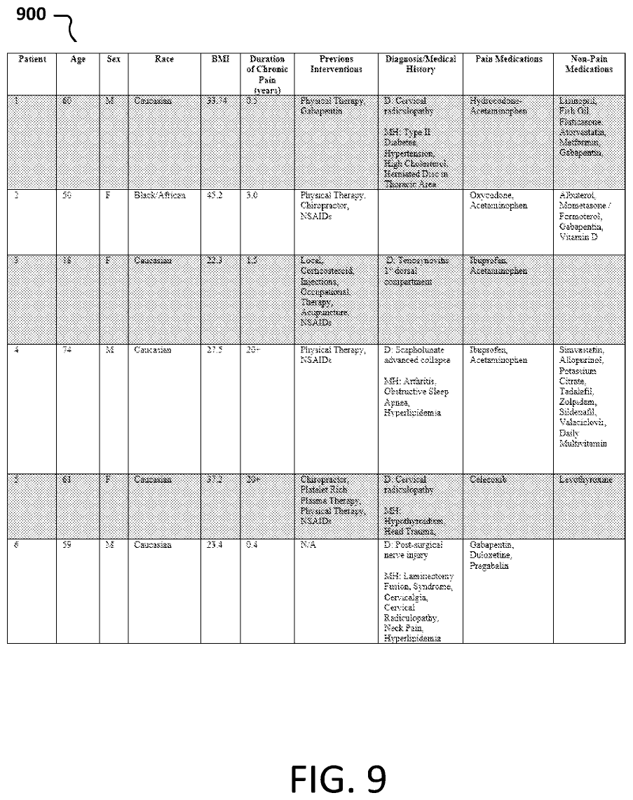

This study included six patients diagnosed with upper extremity chronic pain who met certain inclusion criteria (3 females, three males; age: 59.5±5.5 [median±median absolute deviation (MAD)]; range 18-74 years). The median pre-BCI intervention Visual Analog Scale (VAS) pain rating was 47.5 (±18.5 MAD). Patient demographics, characteristics, and comorbidities are shown in the table 900 of FIG. 9. That is, FIG. 9 is a table showing patient demographics and medical history. In the table: BMI=Body Mass Index, M=Male, F=Female, NSAIDs=nonsteroidal anti-inflammatory drug, D=diagnosis, MH=medical history.

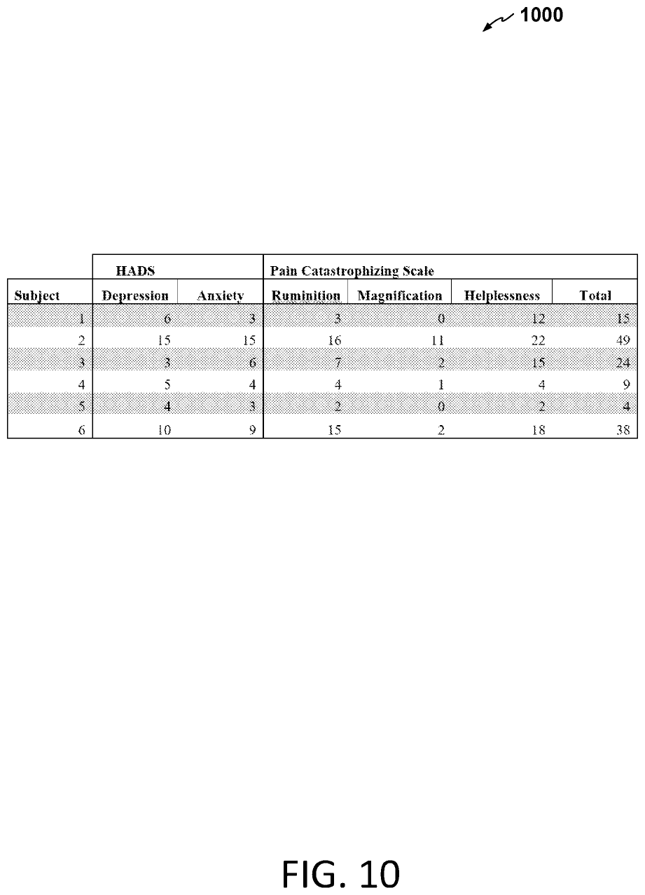

Hospital Anxiety and Depression Scale (HADS) score was 5.5 (±2.0 MAD) for depression and 5.00 (±2.50 MAD) for anxiety. Two of the six patients had depression and anxiety scores above the normal 0-7 range. The total score for Pain Catastrophizing Scale (PCS) was 19.5 (±13.0 MAD). FIG. 10 includes a table 1000 that shows the hospital anxiety and depression scores (HADS) and pain catastrophizing scale (PCS) results for all patients.

Four of the six patients had hypersensitivity in their affected area. One had heat and pinprick hypersensitivity, two had pinprick hypersensitivity, and one had brush and heat hypersensitivity. The location of the pain area and type of hypersensitivity for each patient are shown in Supp. FIG. 1. Three of the patient's affected areas were bilateral, with the average reported pain intensity being the same on both sides. Hypersensitivity, Quantitative Sensory Testing (QST), and conditioned pain modulation (CPM) tests were conducted on the affected side with greater pain intensity on the day of the baseline visit. The results of QST and CPM are reported in FIGS. 1 and 2, respectively. The baseline median scores on the BPI, the primary outcome measure, were 5.25 (±1.63 MAD) for the Pain Severity Score (PSS), and 4.86 (±2.29 MAD) for Pain Interference Score (PIS). The median Neuropathic Pain Symptom Inventory (NPSI) total score before BCI intervention was 17.9 (±9.60 MAD).

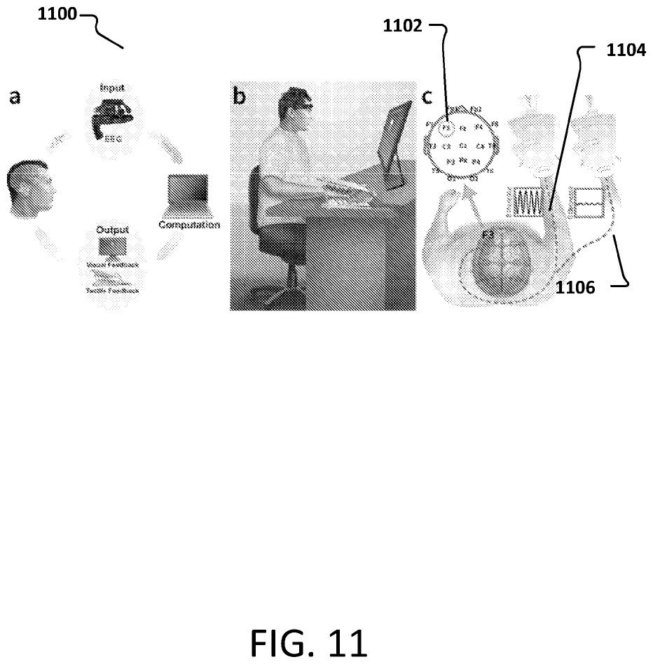

Patients underwent three to five one-half to one-hour BCI training sessions per week over five-six weeks. The intervention duration was specified to be at least five weeks and no more than six weeks. As shown in FIG. 11 (i.e., FIG. 11a and FIG. 11b), the BCI system included a dry electrode electroencephalogram (EEG) headset, a laptop, a custom-made hand vibration stimulation array (HVSA) for functionally relevant sensory feedback, and a video screen for visual feedback. FIG. 11c shows all electrode locations used during recordings, and the integration of the EEG system and the HVSA, where vibrotactile stimulation is delivered to the affected area as a function of patient-generated frontal θ power. More particularly, FIG. 11a shows a system overview schematic of EEG-driven BCI system that includes EEG signals as input and dual visual and vibrotactile sensory feedback. Electrophysiological activity is recorded using a 24-channel dry EEG headset connected to a laptop via a wireless connection. The PC may perform real-time signal processing (i.e., extrapolating frontal midline θ power changes) and command execution (i.e., initiating visual and tactile feedback in response to changes in frontal θ power). Visual feedback was delivered on an external monitor, and tactile feedback was delivered using the custom HVSA. As depicted in FIG. 11b, the system set up includes a BCI system with vibrotactile feedback system enclosing the affected hand. FIG. 11c shows an overview of electrode configuration (top-left) and vibrotactile neurofeedback. Increased F3 θ modulation 1102 during relevant phases of BCI therapy leads to vibrotactile feedback of the affected area 1106. No modulation of θ or decreased modulation of θ leads to no vibrotactile feedback 1104.

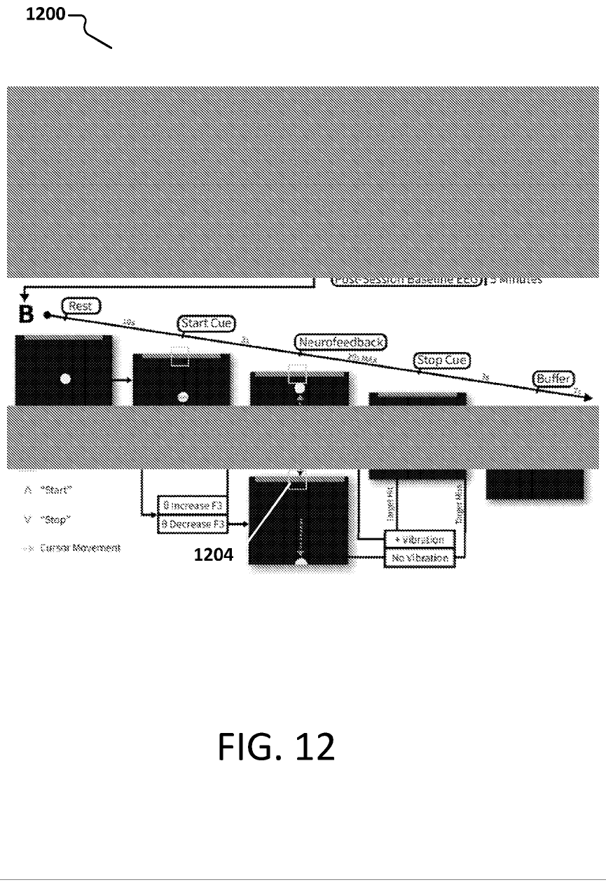

FIG. 12 generally shows an overview of BCI therapy. More particularly, FIG. 12a shows an overall treatment timeline. Solid black shapes indicate the main therapy sections. White shapes indicate specific breakdowns of each main section. FIG. 12b generally depicts an overview of a single BCI trial. The box 1202 shows the numerical stimulus used in early BCI sessions to induce θ modulation. “Start” and “Stop” cues are indicated by arrows. Cursor movement is indicated by the dotted arrow. Trial states progress from left to right. θ modulation direction leads to movement of the cursor during neurofeedback. The neurofeedback period was the only period with a variable duration, with 20 s being the maximum duration of the neurofeedback. If the cursor reaches the green target at the top of the screen, several pulses of vibration are delivered to the affected area over the duration of the “Stop” cue.

Patients meeting inclusion and exclusion criteria attended an initial baseline visit to determine their pain symptom characteristics, record baseline EEG, and perform an initial assessment of task-mediated frontal θ modulation (Supplemental Methods). VAS pain scores were obtained before and after BCI training (main intervention) in each of these sessions. BPI, consisting of pain severity score and pain interference score (PSS and PIS, respectively), and NPSI metrics were obtained once per week via survey after all of each week's training sessions. A specific breakdown of the structure of BCI training sessions is shown in FIG. 12b.

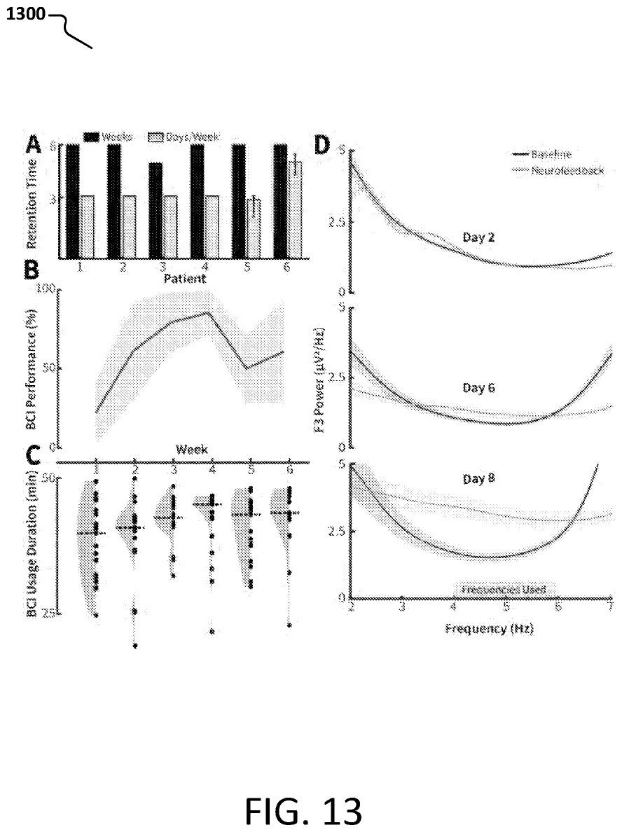

A total of eight patients meeting the inclusion/exclusion criteria attended an initial baseline visit. One patient could not meet the retention criteria and withdrew from the study after the baseline visit (recruitment rate 87.5%, 7/8). One other patient could not adhere to the treatment regimen due to scheduling conflicts and withdrew from the study after the fourth week (retention 85.7%, 6/7). The regimens followed by each of the six patients who completed the pilot study are visualized in FIG. 13a. Five out of the six patients completed six weeks of BCI intervention. One patient completed five weeks. The retention and recruitment rates of this study are comparable to the rates found in larger-scale randomized controlled trials (median recruitment rate 72%, median retention rate 88%) and comparable studies using longitudinal therapeutic BCIs (recruitment range 68.4-80.5%, retention range 55.6%-76.9%).

Turning more particularly to FIG. 13, FIG. 13a shows each patient's intervention time. Black bars represent the number of weeks each patient underwent BCI training. Gray bars represent the mean number of days per week. Error bars represent the range of days per week completed. All patients underwent at least three days of BCI training per week for five weeks. Four of the patients completed three days of BCI training every week. One patient completed three days of training per week for five weeks, then completed two days of training on the sixth week (range 2-3, where only two sessions were completed on the sixth week). One patient completed 4-5 days of training per week for six weeks (range 4-5). FIG. 13b shows a median BCI performance across all participants across six weeks of BCI therapy. The shaded region depicts the median absolute deviation. FIG. 13c depicts Violin plots reflecting session duration across all patients each week. The dotted line represents the median session time across all patients each week. There was no significant difference in session duration between weeks (n=19, 20, 19, 19, 19, 16; Kruskal-Wallis test, df=5, χ2=8.28, p=0.14). FIG. 13d shows exemplar power spectral density plots comparing average θ range power during rest and neurofeedback phases of BCI training for patient. Shaded regions represent standard error. θ modulation increases over BCI training sessions.

As described herein, the median BCI performance across all six weeks of treatment is shown in FIG. 13b. Patients underwent a median BCI training session duration of 43 minutes and completed a median of 1,192 BCI trials over the intervention period. There were no significant changes in weekly session duration throughout the pilot (n=19, 20, 19, 19, 19, 16; Kruskal-Wallis test, df=5, χ²=8.28, p=0.14). Use of the BCI system did not lead to any apparent complications or discomfort for any of the patients. No patients reported any issues or additional symptoms throughout the course of the intervention.

To confirm the feasibility of frontal theta modulation in creating observable differences in task-mediated frontal (F3) θ power, patients performed a serial subtraction mental arithmetic task during their baseline visit, and differences in power spectra were analyzed. These data demonstrate that the serial subtraction task can create qualitative differences in θ power in patients. However, the effect sizes, specificity, and directionality vary. FIG. 13d shows an exemplar progression of power spectral density in θ range for a single patient during BCI training for neurofeedback and rest periods at the F3 electrode. There is a clear qualitative separation of power in θ range between the neurofeedback period and baseline (rest) period of BCI training. Furthermore, the magnitude of the θ modulation during neurofeedback increases with each exemplar BCI training session. In sum, these results reflect the feasibility of the pre-screening task and BCI intervention in producing and detecting modulation in frontal (F3) θ power.

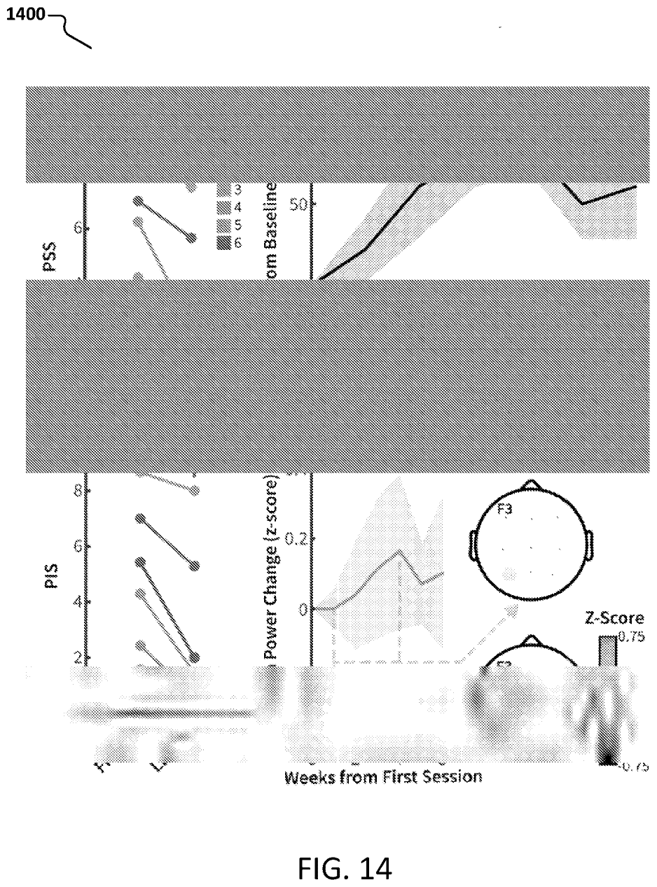

At the group level, for the primary endpoint, there was a significant decrease in PSS and PIS (N=6, Wilcoxon signed-rank, median decrease 1.29±0.25 MAD, p=0.03, q=0.05 and 1.79±1.10 MAD p=0.03, q=0.05, respectively, after BCI intervention. For the secondary endpoint, the decrease in NPSI total score was not statistically significant (N=6, Wilcoxon signed-rank, median decrease 5.25±5 MAD p=0.09, q=0.09). Changes in NPSI and BPI throughout BCI intervention for each patient are visualized in FIG. 14. These results indicate improvements in pain severity and the extent to which pain interfered with day-to-day functions after participation in the BCI intervention.

Turning more particularly to FIG. 14, FIG. 14 includes charts and graphs showing vibrotactile BCI therapy decreases chronic pain symptoms. In FIG. 14a, PSS (N=6, Wilcoxon signed-rank, median decrease 1.29±0.25 MAD p=0.03, q=0.05) and (B) PIS (N=6, Wilcoxon signed-rank, median decrease 1.79±1.10 MAD p=0.03, q=0.05) significantly decreases after the BCI intervention duration. In FIG. 14c, the median BCI performance and VAS rating for all participants across the BCI therapy intervention duration. Across sessions, BCI performance improves while VAS pain rating decreases. The shaded region depicts the median absolute deviation (MAD). In FIG. 14d, the median baseline θ power over the course of BCI therapy at electrode location F3 across participants with either significant pain reduction or consistent BCI performance over the ROC calculated optimal threshold (n=4). Topography plots show median θ power across all electrodes during the first week (upper) and fourth week (lower, where baseline F3 θ was highest). Data were z-scored to baseline θ power, with shaded regions representing MAD.

There were observed differences in BCI performance trends between patients, and thus, it was expected that changes in pain ratings would be different between patients. It is therefore important to acknowledge the individual changes in pain ratings over time and the overall relationship between these trends and frontal θ modulation. Median pain ratings decreased as median BCI performance accuracy increased across all patients over the BCI intervention (FIG. 14c). As discussed herein, the overall trends in BCI accuracy, BCI bit rate, and VAS pain ratings for each patient are shown in FIG. 13. VAS was used to sample pain ratings during independent BCI training sessions, providing a transient metric of pain that was related to electrophysiology and BCI performance. This enabled additional analysis to be conducted to relate electrophysiology, BCI performance metrics, and transient measures of pain. On an individual level, three patients showed a significant decrease in pain over time (n=18, 15, 27, Spearman's ρ=−0.60, −0.75, −0.74; p=0.01, 4.43e⁻³, 1.37e⁻⁴, q=0.02, 0.01, 8.20e⁻⁴). Three of the patients significantly increased BCI performance over the intervention period (n=18, 15, 27, Spearman's ρ=0.49, 0.58, 0.43; p=0.04, 0.03, 0.03, q=0.07, 0.07, 0.07), and one patient began the intervention with a near-perfect performance which decreased mildly over time. Notably, while four of the six participants were classified as either performers (i.e., patients who had a significant increase in BCI performance over time) or VAS pain responders (i.e., patients who had a significant decrease in VAS ratings over time), all six patients had an improvement in primary outcome measures. Taken together, these results suggest a relationship between BCI performance, theta modulation, and pain ratings.

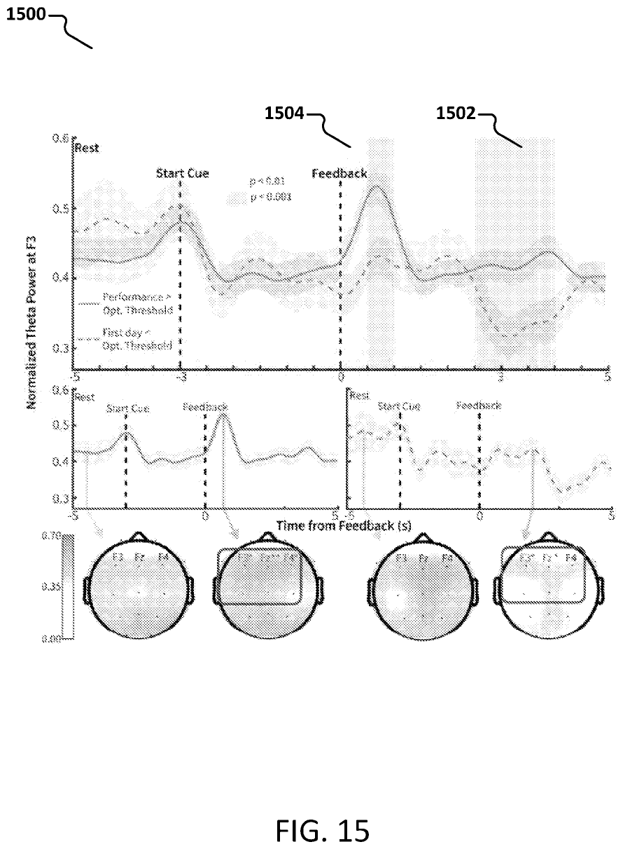

The study included changes in power spectral density across BCI therapy runs. The study was designed to assess whether six weeks of BCI training led to significant resting-state frequency-band power changes. Patients classified as VAS pain responders and performers were those with significant pain decrease over time or with a significant increase in BCI performance over time, respectively. For these patients, resting θ power at F3 increased over the course of therapy, as shown in FIG. 14d. Week four has the greatest median resting θ power across VAS pain responders and performers, which was notably the same week with the greatest BCI performance across all patients (FIGS. 14c and 14d). There was no significant change in the resting power of any frequency band when comparing measures across BCI training sessions for all patients. Topography plots showing qualitative changes in resting power across all electrodes are shown in FIG. 15.

FIG. 15 shows a differential θ modulation before and after BCI control. The top and center portion of FIG. 15 shows patient normalized θ power during sessions with performance above optimal threshold (solid line) and first-day sessions below optimal performance threshold (dashed line). The optimal threshold was determined using ROC analysis to estimate the performance cutoff for BCI sessions with the greatest likelihood of significant θ modulation during neurofeedback. Black dashed lines indicate the onset of the start cue and neurofeedback spans. Shading 1502 indicates p values at durations without standard error overlap. Another portion 1504 shows duration without overlap between traces (500 ms window, n=60, p=0.006). Section 1502 indicates durations of statistically significant differences between time traces (500 ms window, n=60, Wilcoxon rank sum test, p<0.0005, Bonferroni Corrected). The line shows averaged power values across each within-subject average, with the shaded region showing average standard error. The bottom portion of FIG. 15 shows a topography of the normalized θ power during baseline and peak normalized θ during neurofeedback before and after BCI control. Theta power increases are localized to frontal channels during high BCI performance.

In order to identify the specificity of θ modulation and investigate whether frequency-specific modulation was different between high BCI control and the absence of BCI control, the progression of frequency-specific power was compared during BCI training trials. FIG. 15 shows the median progression of normalized θ power across BCI training trials. The normalized θ power, averaged across all BCI sessions, was compared between high BCI performance and the absence of BCI control. These results exemplify the qualitative difference in θ power modulation specificity between sessions where patients did not yet acquire BCI control with sessions where subjects achieved BCI control. There were significant differences between the two power traces only during neurofeedback for F3 θ power, specifically 0.5 s to 1 s and 2.5 s to 4 s relative to neurofeedback onset. For a frequency band at F3, there was a significant difference in power from −4 s to −3 s and 3 to 3.5 s. For β frequency band at F3, there was a significant difference from 2.5 s to 3 s (For θ, α, and β, 500 ms window, n=60, Wilcoxon rank sum test, p<0.0005, Bonferroni corrected). There were no significant differences between power traces for δ and γ frequency bands. Traces for all power bands, including topography plots at each time point, are provided as videos in supplementary materials (Supp. Movie 1, Supp. Movie 2, Supp. Movie 3, Supp. Movie 4, Supp. Movie 5). Qualitatively, θ modulation was specific to the frontal region during high BCI performance, indicating the specificity of neuromodulation. Notably, there was a consistent decrease in a power during the neurofeedback period, irrespective of performance, which may indicate patient engagement in mental tasks.

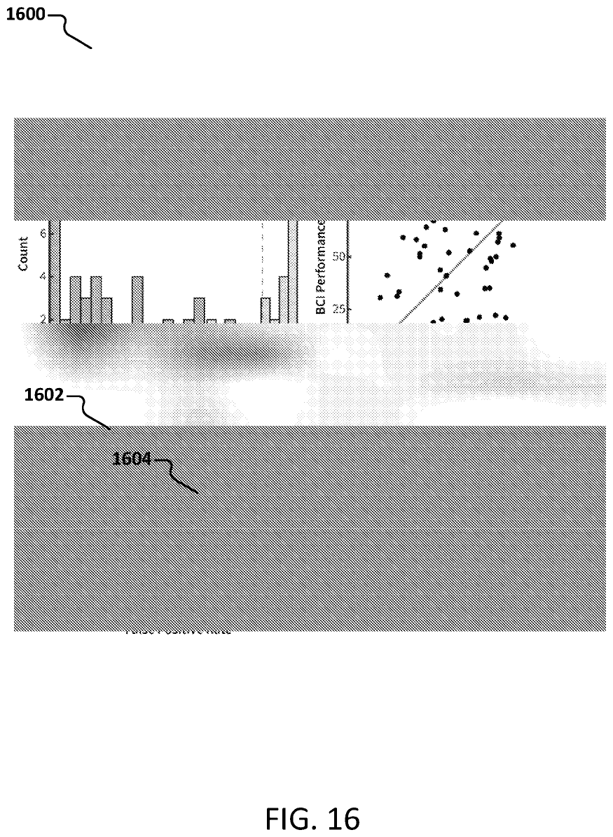

To confirm that BCI performance was a proxy for θ modulation, and thus further validating the feasibility of this intervention, the distribution of BCI training sessions was plotted across all patients by performance with and without a significant increase of each frequency band at F3 during neurofeedback. Receiver operating characteristic (ROC) analysis revealed an area under the curve (AUC) of 0.73 for δ, 0.90 for θ, 0.84 for α, 0.76 for β, and 0.78 for γ, as shown in FIG. 16.

FIG. 16 shows graphs illustrating the relationship between θ modulation, BCI performance, and pain relief. The top of FIG. 16 shows a distribution of all BCI sessions binned by performance. Shaded bars 1602 represent sessions where participants significantly increased their θ power during the neurofeedback period relative to the pre-intervention baseline (Rank-Biserial Spearman's Correlation, cyan: p<0.05 and ρ>0). The dashed line 1604 shows the optimal threshold calculated via ROC analysis. The bottom portion of the FIG. 15 shows an ROC curve of the patient performance distribution. AUC of 0.90 and threshold of 0.83 or 83% for optimally classifying sessions with significant θ modulation. FIGS. 16b and 16c θ modulation correlates with BCI performance as well as pain relief. FIG. 16b shows the relationship between BCI performance and θ modulation (n=106, Spearman's ρ=0.67, p<0.001) across all subjects and sessions. FIG. 16c shows the relationship between pain relief (percent pre-intervention baseline) and θ modulation (n=106, Spearman's ρ=0.32, p=0.001, Bonferroni Corrected alpha=0.005) across all subjects and sessions.

BCI performance was the greatest predictor of power increase for θ frequency band at the BCI classification electrode F3. Also, the distributions of training session performance tended to congregate at either lower or higher performance values, with higher BCI performance corresponding to significant θ power increase during neurofeedback. This trend was not observed for any other frequency band. Sessions with significant F3 θ increase during neurofeedback also accounted for the greatest number of sessions with significant power increases across all frequency bands. There were 9 sessions with a significant δ increase, 38 with a significant θ increase, 23 with a significant α increase, 9 with a significant β increase, and 17 with a significant γ increase, as shown in FIG. 16. θ modulation strength was positively correlated with BCI performance (n=106, Spearman's ρ=0.67, p<0.001, as shown in FIG. 16b.

After confirmation that the BCI intervention was able to specifically serve as a proxy for θ modulation, the relationship between pain relief and θ modulation was then examined. There was a direct positive correlation between pain relief and θ modulation increase during BCI training (n=106, Spearman's ρ=0.32, p=0.001, Bonferroni Corrected alpha=0.005, FIG. 6c). This relationship was not significant for any other power band (Supp. FIG. 6). The correlation for both pain ratings acquired before and after each BCI training session (n=106, Spearman's ρ=0.29 and 0.32, p=0.0009 and 0.001, respectively, Bonferroni Corrected alpha=0.005). A correlation was found between pain relief and F3 θ modulation strength in both cases, which alludes to both direct pain relief as a function of each day's performance, as well as an overall pain reduction as a function of increasing BCI performance throughout the duration of the therapy.

In this six-week pilot study of patients with chronic upper extremity pain, a vibrotactile BCI therapy was used to increase frontal theta power and measure patient-reported changes in pain symptoms. Feasibility goals of patient retention and treatment protocol adherence were met. The results further indicate that this intervention reliably modulated frontal theta power and demonstrated feasibility in reducing pain. Specifically, this study met its proposed endpoint of a significant decrease in both pain interference score and pain severity score after BCI intervention in this group of patients. This result may allude to the generalizability of this treatment, as this patient group had heterogeneous demographics, initial symptoms, and trends in VAS pain ratings. Throughout the vibrotactile BCI therapy, the magnitude of frontal θ modulation was significantly and positively correlated with pain relief as measured by VAS. This was unique to frontal θ rhythms (F3 location) and was not present in any other frequency-specific power bands, indicating the specificity of θ modulation in pain relief. These results support the initial hypothesis that a non-invasive vibrotactile BCI device can feasibly target and reinforce brain activity associated with pain relief. These results warrant future work to further clinically validate this approach and to better elucidate the mechanism that underlies how cortico-sensory coupling impacts chronic pain patient populations.

The experience of pain and pain relief has been shown to involve a complex neuronal network, often referred to as the pain neuromatrix. This model suggests contributions of bottom-up and top-down neural regulation of pain experience, including features beyond pain sensation such as emotional and salient components. In neuromodulation studies using transcranial magnetic stimulation (TMS) or transcranial direct-current stimulation (tDCS), regions associated with top-down modulatory mechanisms of pain have been targeted, yielding successful reductions in pain symptoms. TMS, applied to the dorsolateral prefrontal cortex in multiple experimental and chronic pain studies, led to pain reduction. Similarly, various studies using tDCS of the prefrontal cortex have previously been shown to reduce pain. It is hypothesized that these techniques target the activity of pain perception and modulation areas, including the cingulate cortex, insula, amygdala, and thalamus. These regions have previously been shown to play a major role in the top-down modulation of pain and are perturbed during chronic pain. More recently, the electrophysiology of pain relief in acute and chronic settings has demonstrated that EEG frontal θ power increases are associated with the remittance of pain. Source localization of these brain signals converges with previously identified anatomic sites from TMS, tDCS, and functional imaging studies. These data motivated the electrode (F3) and frequency band θ configuration of this BCI system. The positive pilot results of this study support that the reinforcement of θ activity in this region may enhance the top-down capacity of critical sites such as the dorsolateral prefrontal and cingulate cortex to dampen noxious and aversive sensory perceptions.

Using a BCI to enhance the cortical physiology of pain relief as a therapeutic strategy is unique from prior approaches. Most studies using non-invasive neurofeedback for chronic pain have targeted pathological profiles of the sensory perception of pain, specifically by attempting to decrease activity associated with nociceptive processing. A review describes targeted neurofeedback systems, showing that the majority of existing work using neuromodulation for chronic pain has reinforced or inhibited EEG power at a variety of frequencies mainly over somatosensory areas (C3, Cr, Cz, T3, T4, P3, and P4 electrodes), yielding variable success in relieving symptoms of chronic pain. The strategy for frequency band modulation was typically to increase α power (8-15 Hz), decrease β power (18-22 Hz), and occasionally decrease θ power. Only one study reinforced activity at frontal electrodes, but this was α at FP1. These studies approached the problem of chronic pain by targeting pathological activity patterns associated with the encoding of pain over somatosensory areas. While a logical approach, the pathological profiles of pain are highly variable and can differ depending on the type of chronic pain. The approach targeted reinforcement of activity patterns associated with pain relief (i.e., left-frontal θ power increase), which was shown to be present in both relief from experimentally induced tonic pain and relief from chronic pain. Given the substantial similarities of pain relief cortical physiology between these two studies with very different pain experiences, this physiology may be less variable across patients experiencing pain and enable a more broadly applicable BCI-mediated neurofeedback strategy.

Beyond the choice of cortical location and frequency band, the type and character of feedback are also critical. Prior BCI systems applied to chronic pain have used visual feedback that changed with brain signals associated with somatomotor intentions. For a system to be effective in neurorehabilitation of pain circuits, visual feedback is not likely to be optimal. To optimally induce remodeling via Hebbian learning, it is important to temporally couple a desired cortical physiology with functionally relevant sensory feedback to the affected anatomic location. For example, in the setting of chronic stroke, it was critical to couple brain signals associated with motor intentions with proprioceptive kinematic feedback provided by a robotic exoskeleton to achieve a functional improvement. In the setting of chronic pain, signals associated with pain relief (e.g., increased frontal θ power) were coupled with pain alleviating vibrotactile sensory stimulation in the distribution of pain, to enable neural remodeling to occur. To this end, in addition to providing visual cues of the patient's performance in controlling their frontal θ power, patients also received sensory-relevant vibrotactile feedback with stimulus intensity proportional to the magnitude of θ modulation. Previous evidence has shown that tactile feedback provides sensory-relevant feedback and can improve BCI control. Similarly, the use of vibrotactile feedback, which has previously been shown to lead to transient pain relief, can act as a potent reinforcer to the central physiology of pain relief. From a Hebbian standpoint, the use of a BCI is especially salient because transient pain relief co-occurs precisely in time with the θ changes in the frontal lobe. While an important consideration for pain, this general configuration of BCI-mediated cortical-sensory coupling has yet to be optimized. It is unclear what are the optimal vibrotactile parameters in terms of intensity, timescale, and vibration frequency to best support central neural remodeling to lead to long-term pain relief.

While the central results of this pilot study are clinical improvements with the use of the system, there are also intriguing neurophysiology findings that support the notion of neural remodeling occurring. Specifically, baseline θ power (in the absence of task) changed over time, primarily in the left hemisphere. This change occurred both in frontal and central regions (EEG electrodes F3, C3, P3). There is prior evidence that resting-state dynamics of θ rhythms have been considered a biomarker of BCI-mediated changes in the brain. In the setting of BCI-induced motor rehabilitation in chronic stroke, the degree of change in resting-state θ rhythms was closely correlated with the degree of motor improvement. Similarly, those patients with significant improvement in their pain demonstrated notable changes in their fronto-central baseline power. While too early to make definitive conclusions, these θ power changes merit future exploration as a potential EEG biomarker for assessing the impact of BCI-mediated pain therapy.

While the results demonstrate the potential of a BCI-mediated neuromodulation therapy for chronic pain, there are several limitations that merit consideration. Primarily, this clinical pilot included only six patients making more generalized claims limited. This study also lacked a control group to differentiate between the effect of θ-driven vibrotactile stimulation and placebo effects, particularly considering the setting of in-person visits to the research facility. In the future, it will be important to increase the number of patients to power the study adequately and include an additional pseudo-neurofeedback control group which will receive the visual and vibrotactile feedback, in comparable dosages, without any correlation to brain activity modulation. This will better define the role of frontal θ modulation in reducing pain, and whether coupling the vibrotactile and visual neurofeedback is effective in mitigating pain symptoms. Further, this control condition will be able to decouple the effect of frontal θ reinforcement and vibrotactile feedback over a longitudinal intervention period on observed therapeutic outcomes. A larger study will also be necessary to define electrophysiological biomarkers such as general changes in power, connectivity, and phase-amplitude coupling, which have previously been shown to be associated with pathology in chronic pain. The dosage (i.e., how long patients should be treated) and the durability of the treatment (i.e., how long the effect lasts) will need to be defined in future studies. Future iterations of this BCI will also involve the refinement of parameters, including feature discrimination and feedback optimization, to maximize therapeutic effect. As an initial pilot study, the approach to feature selection and feedback parameterization was simple. In the future, machine learning approaches can be used in several ways to increase BCI-literacy and therapeutic output. Variance in spectral properties between patient EEG implies feature weights can be modified in order to best tailor BCIs to individual patients. Specifically, classification training can be used to select the best spectral targets for reinforcement in order to maximize therapeutic benefit. Given the importance of feedback in BCIs, machine learning methods can also be used to converge on vibrotactile stimulation feedback parameters to maximize reinforcement of frontal θ power and therapeutic outcome.

This pilot study aimed to assess the feasibility of a frontal θ driven BCI mediated vibrotactile therapy for chronic pain. The effectiveness of frontal θ reinforcement using the BCI, and that reductions in patient pain severity and pain interference scores can be achieved through use of the system.

For the scope of this pilot study, intervention feasibility was assessed on various criteria. Patient adherence and retention were reflected in participation and treatment frequency. Patients were instructed to attend BCI training sessions for a minimum of three days a week over the intervention duration. Patients were encouraged to participate in each session for 30-45 minutes but were accommodated for scheduling conflicts. During treatment sessions, patients were instructed to allocate as much attention as possible to the BCI training task (main intervention), to turn off communication devices, and to talk only during the allotted break periods. Those unable to adhere to this regimen during the intervention were given the option of restarting the treatment (i.e., restarting the protocol from the first week). The inability to adhere to the treatment schedule resulted in withdrawal from the study. Data from patients who did not complete five weeks of training were not analyzed. Successful patient retention was defined as completing at least five weeks of intervention for at least three days a week. Weekly session duration was assessed to determine patient adherence and retention. Intervention safety, risk, and comfort were determined by patient-reported feedback after each BCI training session. BCI patients were instructed to verbalize any discomfort they experienced and whether they felt any part of the intervention was unreasonable.

The success of this pilot's objectives and hypotheses are predicated on the intervention's design in successfully inducing modulation in patient θ power during BCI training. Two main steps were used to assess the feasibility of the intervention in producing and detecting modulation in left frontal θ. First, a pre-screening frontal θ modulation task was implemented. This task aimed to assess each participant's ability to modulate θ activity and whether this modulation could be detected via analysis. Second, the qualitative changes in θ power during consecutive days of BCI training were assessed and compared to BCI performance scores and pain reduction. BCI performance was evaluated to serve as a proxy for θ modulation strength and whether the degree of θ modulation was related to changes in pain ratings.

Patients completed self-report questionnaires, underwent cognitive testing, baseline brain activity recordings, and screening for task-specific θ modulation prior to any BCI-intervention. Specifically, participants rated their pain intensity on VAS, completed BPI, NPSI, and the PCS. In addition, HADS was used to determine patient anxiety and depressive symptoms. QST was performed to assess patients' somatosensory profile. The EEG recordings took place in a quiet study room with minimal outside disturbance. Participants were comfortably seated in a stationary chair and were asked to limit their movements during brain activity recordings. The details of the methods used to obtain these metrics are described in the Supplemental Methods.

The BCI system consisted of a DSI-24 EEG recording system connected to a laptop via wirelessly, enabling easy and comfortable measurement of high-fidelity EEG signals in a laboratory environment with minimal setup time. The HVSA provided real-time vibrotactile neurofeedback, and a monitor provided visual feedback in the form of a vertical cursor task. A general-purpose, open-source modular software was used to manage data acquisition, real-time signal processing, application execution, event logging, and system synchronization. This device consisted of a custom-designed adjustable frame able to fit most hand sizes, with two (upper and lower) grids of 24 small vibrating motors contacting both sides of the hand. This device executed vibration commands via a controller connected to the PC.

A real-time spatial and spectral filter was applied to acquired EEG data to extract frontal-midline θ power. A one Hz high pass and 50 Hz low pass filter were applied in real-time to avoid the influence of artifacts from line noise or channel drift on BCI classification accuracy. Signals were common average re-referenced, then spatially filtered such that F3, Fz, and F4 electrodes were selected as feature input channels. θ power was calculated in real-time by an autoregressive (AR) spectral estimation using a 500 ms sliding window. The AR estimation was selected as it provides a computationally efficient means for real-time spectral estimation and has been shown to have high spectral resolution even for shorter data segments. 4-6 Hz power was defined as the as the driving spectral feature. 4-6 Hz was chosen over 4-7 Hz to minimize the effect of a band variance on the spectral filter. Previous evidence has suggested task-specific and resting-state variance of a power both within and between populations of subjects. Further, the proximity of commonly defined a frequencies to the desired spectral feature of θ could have potentially influenced the specificity of the spectral estimations due to spectral leakage. To avoid these confounds, frequencies closest to either θ or a as part of the real-time feature extraction were not included. This real-time processing method enabled computationally efficient processing in order to minimize the latency between real-time processing of electrophysiology and execution of neurofeedback commands.

To define baseline θ power for real-time classification during BCI training for each patient, a zero-mean distribution of the subject's resting θ power at the F3 channel was generated. This was done during a set of ten adaptation BCI trials, where the adaptation function was enabled. This function optimized normalization values for calculating relative changes in θ power by determining a zero-mean value for θ power during rest, and an arbitrary gain value which aided in magnifying output commands. This gain value was also optimized such that patient eyeblink artifacts, which lead to transient increases in δ−θ power, did not significantly influence BCI performance metrics (primarily by regulating the rate of trial completion, such that consecutive eyeblinks alone could not lead to trial success). After ten of these adaptation trials, a set zero mean value was uniquely used for the remainder of the BCI therapy for each patient. The output of the classifier was thus a θ power value normalized to the baseline zero-mean value, multiplied by the determined gain value: output=(input−zero mean value)*adaptive gain value. The output value was used to drive neurofeedback, with positive values indicating increases in θ power at F3 and negative values indicating decreases. The overall magnitude of the output value was reflected in the velocity of the visual cursor and the intensity of the vibrotactile feedback, as described herein.

Patients underwent at least three one-hour BCI training sessions per week for at least five weeks, but no more than six weeks (intervention duration). This duration and weekly session number were selected based on intervention durations of previous longitudinal BCI or neurofeedback therapies. VAS pain scores were obtained from patients upon arrival to the study room and immediately after each day's BCI training session. Baseline recordings were obtained via EEG during the pre-BCI training period each day (5 minutes), during the BCI training session (up to 45 minutes), and during each day's post-BCI training session (5 minutes). During baseline recordings (pre and post BCI training), patients were asked not to perform specific mental tasks and to remain calm and relaxed while remaining awake. A fixation cross was presented on the display, and patients were asked to fix their gaze on the cross. Patients were instructed to blink at a natural rate, ensuring that they did not blink inconsistently between parts of the BCI trials. After the pre-BCI training recording, patients were asked to place their affected hand into the HVSA.

BCI training consisted of three 15-minute blocks. Each trial consisted of a ten-second baseline period, a three-second start cue period, a 20-second limit neurofeedback period, a three-second stop cue, and a seven-second post stop-cue pre-baseline buffer period. The subjects were presented with a yellow on-screen cursor which remained stationary at the center of the screen during non-neurofeedback periods. At the top of the screen, a green rectangle indicated the target and remained on top of the screen during all periods. The green rectangle served as the target and would always appear at the top of the screen in the same location. During the start cue, the word “Start” was presented in the center of the cursor, and a random prime number between 3 and 19 appeared in the center of the top rectangle in cyan. This prime number remained in the rectangle for the duration of the neurofeedback period, as an option for patients to use serial subtraction as the driving task for frontal θ modulation if needed. Additional methods regarding the role of the prime numbers are provided in Supplemental Methods. After three seconds, the word “Start” would disappear, and the neurofeedback period would begin. During the neurofeedback period, the center cursor would either move up if the subject increased their left frontal-midline θ power above baseline or move down if their left frontal-midline θ power decreased below baseline average. The speed of cursor movement corresponded to the magnitude of increase or decrease (see description of intervention). During neurofeedback, the HVSA would stimulate the participant's affected area when there was an increase in θ power. The intensity of the vibration corresponded to the magnitude of θ power increase. The vibration was pulsed at a random frequency selected from 4, 5, 6, or 7 Hz. If the participant was able to reach the target by moving the cursor to the top rectangle within the allocated 20-second period, the stop cue would initiate, and a burst of vibration at maximum intensity was delivered for the duration of the three-second stop period. If the participant could not reach the target within the allocated time limit, the stop cue would initiate with no vibration delivered. The stop cue consisted of the cursor returning to the center of the screen, and the word “Stop” was displayed in the center of the cursor. The seven-second buffer period allowed for any brain activity changes resulting from the stop period and the neurofeedback period to wash out such that the activity recorded during the baseline period was unaffected. FIG. 2b shows the design of a single trial within a training session. At the end of each week on Friday, patients received an email containing a survey. The survey was used to obtain self-reported BPI and NPSI metrics each week outside of the experimental environment.

Electrophysiological activity recordings throughout the therapy duration were carried out using dry-surface EEG. Primary and secondary outcome measures were self-reported by patients. EEG was recorded using a 24 wireless dry electrode headset in an international 10-20 system. EEG was referenced to the Pz electrode and sampled at 300 Hz with a ground electrode placed on the earlobe. Electrode impedance was maintained below 10 kΩ. The primary outcome measure was the BPI PIS sub-score. Secondary outcome measures were the NPSI total score (50 points total, consisting of sub-scores of 10 points each for burning pain, pressing pain, paroxysmal pain, evoked pain, and paraesthesia/dysaesthesia, where higher scores reflect more severe symptoms of a specific quality) and BPI PSS sub-score, which were recorded at the end of each week for each patient. These outcomes, especially BPI, are considered robust and reliable outcome measures for trials in patients with chronic pain, where higher scores indicate worse or more severe symptoms related to the quality and intensity of pain (PSS, 0-10 with 0=no pain and 10=worst imaginable pain), and the degree that the pain symptoms interfere with everyday life (PIS, 0-10 with 0=no interference and 10=interferes completely). These metrics have been adapted to a variety of diseases and types of pain. For reference, previous work has reported that 1-point changes in PIS reflect a typical 0.5 standard deviation change, and is a benchmark for minimal clinical importance, and improvements of 2 points or greater reflect moderate or meaningful changes. For pain severity, recorded as PSS, 10% point changes were considered minimally important, while reductions of 30% or more were considered to be clinically notable. These metrics were acquired via patient response on a survey emailed to each patient at the end of each week. Survey outreach was handled by the database/survey management software. VAS scores (additional secondary outcome, where 0=‘no pain’ and 100=‘worst pain imaginable’) were acquired from patients before and after each BCI session and used for secondary analysis to relate changes in pain ratings, BCI performance, and electrophysiological features. Processing of electrophysiological data and statistical analysis was conducted.

EEG data were preprocessed by first applying a 1 Hz high pass and 40 Hz low pass Butterworth filter to the signal. Next, high amplitude signal artifacts were removed by thresholding above each subject's manually determined eyeblink artifact threshold. This was done for each data file qualitatively by manually identifying the largest magnitude eyeblink artifact and then setting the threshold above this value. The threshold was set above the magnitude of the eyeblink artifact because independent component analysis (ICA) was used to remove eyeblink artifacts, which minimized the amount of electrophysiological activity removed from the data. Signals containing samples above this threshold were deleted and then smoothed using a five-point moving average interpolation. The signal was then common average re-referenced, and ICA was performed to remove eyeblinks, eye saccades, lateral eye movements, muscle-related, and cardiac-related artifacts from the data.

Band-limited amplitude time series was extracted by applying a Butterworth with frequency cutoffs as follows: 1-3 Hz for delta (8), 4-7 Hz for θ, 7-13 Hz for alpha (α), 13-30 Hz for beta (β), and 30-40 Hz for gamma (γ). Power envelopes for each signal were calculated by squaring the absolute value of the Hilbert transform of the time series. The mean power envelope within each relevant timeframe was used for overall power calculations.

For electrophysiological comparisons within and between subjects across multiple days, power values were normalized to baseline recordings by z-scoring all power envelope time series to power envelope from EEG data recorded before any BCI intervention.

For analysis of continuous, relative power changes within single BCI sessions, a time-frequency analysis was conducted by time locking pre-processed signals acquired from each session across each subject to trial start and neurofeedback onset. Time-frequency was determined by the mean Fast Fourier Transform of all trials with Hamming window tapering. Time-frequency signals were binned into power bands as described above, then normalized to the minimum and maximum power values within each session, exemplifying the spatial-temporal modulation of different frequency-specific activities within each BCI training session.

For analysis of spectral properties between conditions, specifically, to visualize qualitative differences in power spectral density plots, Welch's power spectral density estimate was used with a Hamming window equal to ten percent of the epoch duration. This analysis was applied to demonstrate qualitative differences in θ power modulation during the pre-screening mental arithmetic task and BCI training.

Statistical tests were conducted using non-parametric methods. None of the implemented statistics were used for concluding clinical or mechanistic outcomes. Rather, the tests employed for this study aimed at quantitatively supporting the feasibility and potential of the device for larger, placebo-controlled clinical trials and for identifying potentially relevant mechanisms evoked by BCI intervention that justify further investigation. For correlation analysis conducted between pain intensity and treatment time, BCI performance (measured as BCI accuracy: #trials goals reached/#total trials) and treatment time, BCI bit rate and treatment time, θ power modulation and BCI performance, and θ power modulation and pain relief, Spearman's correlation was used. BCI throughput (bit rate) was calculated using Fitts's metrics in bits per second as an alternative means of measuring BCI performance in addition to accuracy. Throughput (bits per second) was calculated as the cursor task's index of difficulty which was equal to the log 2 (1+238) over the time to reach the target for each trial. 238 was the total distance the cursor traveled in pixels to reach the goal. For trials where the goal was not reached, the time to reach the target used for the calculation was 20 seconds, as this was the maximum duration participants had to reach the goal. Patients with a significant increase in BCI performance accuracy over time were classified as performers. Patients with significant decreases in pain over time were classified as VAS pain responders. To determine the significance and directionality of power modulation from baseline, a non-parametric rank-biserial Spearman's correlation was used. This method enabled correlation between continuous ranked variables (i.e., EEG data from two conditions) and a dichotomy (i.e., a generated data vector of −1 s and 1 s corresponding to the lengths of either EEG data conditions). This method was chosen over a Wilcoxon rank-sum test as it reflected the directionality and magnitude of significantly different frequency-specific power. To compare group-level changes in the outcome measures, a two-sided Wilcoxon signed-rank test was used. For statistical comparisons of power differences at single channels between conditions, a two-sided Wilcoxon rank-sum test was used. To account for multiple comparisons for these statistical tests, a Benjamini and Hochberg procedure was used to calculate the false discovery rate (q-value), as this test has shown to be more conservative and better fit for fewer comparisons. Due to the small study size, the median was used as the central tendency value for comparisons across all subjects, and the median absolute deviation (MAD) was used for variance. The mean and standard error were used for comparisons of electrophysiological data consisting of multiple trials. For detecting whether there were significant differences between weekly intervention participation time, a Kruskal-Wallis test was used. Repeated measurements were taken across all participants for both clinical and electrophysiological variables.

ROC analysis was performed to assess whether BCI performance was an accurate proxy for θ modulation. For each session, it was determined whether significant θ modulation was achieved by performing a rank-biserial Spearman's correlation between pre-BCI intervention baseline θ power and each session's neurofeedback period θ power at F3. Baseline epochs and neurofeedback epochs were the same lengths. Sessions with successful θ power increases as those with a p<0.05 and Spearman's ρ>0 were defined as a success. ROC analysis was performed on the distribution of all sessions, including sessions with or without significant increases in θ power at F3. An optimal performance threshold for identifying a BCI performance value most likely to reflect θ power increase at F3 was determined by maximizing the true positive probability (i.e., session performance truly reflects significant θ power increase) and minimizing false positive probability (i.e., session performance reflects significant θ increase without actual increase). Sessions with high BCI performance were defined as sessions with performance above the optimal threshold calculated with ROC analysis (83%). Sessions with no BCI control (naïve sessions) were defined as each patient's first BCI training session with performance below the optimal threshold.

For comparisons of normalized band-power differences between traces generated by these groups, data within 500 ms bins was compared using a two-sided Wilcoxon rank-sum test. Permutation tests (1000 iterations) were performed for all correlations, a biserial correlation analysis. The relationship between magnitude θ increase (Spearman's ρ) was compared with BCI performance and pain relief using Spearman's correlation. Significance levels were Bonferroni corrected for these tests.

An embodiment may include a theta-controlled vibrotactile brain-computer interface to treat chronic pain. A corresponding study mapped a participant's affected extremity for areas of spontaneous pain and thermal hypo/hypersensitivity was done by exposing the area to a 20° C. cold stimulus, 40° C. warm stimulus, brush, and pinprick. Quantitative Sensory Testing (QST) was performed at baseline using an abbreviated version of the German Research Network on Neuropathic Pain (DFNS) protocol to determine differential sensory profiles of each participant's affected (most painful) and unaffected (non-painful) regions. This was used to assess warm and cold detection (WDT and CDT) thresholds, heat and cold pain thresholds (HPT and CPT), mechanical detection and pain thresholds (MDT and MPT), presence of wind-up (enhanced temporal summation) to pinprick, vibration detection thresholds (VDT), and pressure pain threshold (PPT), and conditioned pain modulation (CPM). The Thermal Sensory Analyzer was used to determine WDT, CDT, HPT, and CP. A set of standardized Semmes-Weinstein monofilaments (0.25, 0.5, 1, 2, 4, 8, 16, 32, 64, 128, and 256 mN) was used to assess MDT for each participant's affected and unaffected area. A set of calibrated blunt metal probes with applied force 8-512 mN was used to determine the MPT. Wind-up ratio (WUR) was assessed using a #6.10 von Frey filament (980 mN) and calculated as the ratio between pain intensity elicited by a train of ten 1 Hz stimuli versus a single stimulus value was calculated. Each participant's frequency of vibration detection threshold was determined using a clinical tuning fork (64 Hz, 8/8 scale). A handheld algometer was used to determine each participant's PPT following continuous pressure application at a 0.5 kg/sec rate. The test sites used for affected and control sites were along the same muscle. A cold-water bath and the Thermal Sensory Analyzer were used to assess each participant's CPM. CPM was evaluated by determining the change in elicited thermal pain rating before versus during exposure to a conditioning stimulus (cold water bath maintained at 12° C.). The test stimulus was determined as the probe temperature, which elicited a pain intensity of 50 on a 0-100 NRS in each participant. First, the Pain-50 stimulus was applied, and the subject-reported pain intensity (NRS) evoked by the stimulus was documented. This procedure was repeated twice. Next, the Pain-50 stimulus was applied during the last 30 seconds of a 60-second contralateral cold conditioning, and the participant was asked to report the pain intensity elicited by the thermal probe. This was repeated twice. The difference between the reported pain intensity of the stimulus before and during conditioning was the CPM magnitude. A CPM <0 implies efficient descending pain modulation.

Prescreening including training subjects to control a BCI using frontal-θ power, a task was provided to initiate modulation of this pattern. An EEG was collected during a serial subtraction mental arithmetic task to assess each participant's characteristic θ modulation profile. Mental arithmetic, calculations, and mental tasks increase frontal midline θ activity. This observation was used to pre-screen participants for task-specific modulation. This strategy was used for all participants to induce initial θ modulation, which could be used to exert BCI control. Baseline EEG data were collected for five minutes before the experiment. The task design consisted of two 25-trial blocks. Before assessment recording, the subjects were given randomly generated three-digit multiple of 50. This number would be used for the serial subtraction task. Each trial consisted of a five-second task period where participants were asked to engage in serial subtraction and a five-second baseline period where subjects were asked to fix their gaze on a fixation cross presented at the center of the display without performing any mental tasks. During the task period, a pseudorandom prime number between 3 and 19 was selected and presented to the participant in the middle of the screen. The participants were asked to serially subtract that prime number from the three-digit multiple of 50 until that number disappeared and the baseline fixation cross was presented. Offline analysis was conducted to quantify the degree of θ modulation and quantify the spectral properties of their activity modulation.

Specific instructions given to patients when engaging in BCI task. Participants were informed about the context of the BCI intervention during the baseline visit, and instructions were reiterated briefly at the beginning of each BCI training session. Participants were informed that the research group had identified an EEG biomarker (frontal θ power) that may have therapeutic implications for chronic pain symptoms and that the present study wanted to evaluate whether BCI could be leveraged to target and reinforce this activity. Participants were asked to watch an informative video which provided instructions and context to what the BCI intervention would entail. They were informed that during BCI training, successful increases in frontal θ power would result in ascending cursor movement towards a goal on the screen, and concurrent vibrotactile stimulation of the hand on their affected limb. Patients were given examples of previous methods identified in the literature, where increases in frontal θ power have been observed. Patients were then told that serial subtraction would be the main task recommended by the research group to begin modulating frontal θ power. They were told that during BCI training, a series of prime numbers would appear on the goal, and they would have the option to use these prime numbers to guide a serial subtraction task. They were told that serial subtractions involved repeatedly subtracting the same number (in this context, the prime number that appeared on the screen) from a provided number (which was a randomly generated multiple of 50 between 1000 and 2000 and provided to the patient before each BCI session). The patients were also provided with alternative examples, including meditation and other cognitive control-related tasks. The patients were encouraged to try various methods and that they would always have the option to default to the serial subtraction tasks if needed. Participants were told that any kind of cognitive task leading to vibrotactile feedback and moving the cursor to the goal would be an appropriate means to increase frontal θ power.

Spectral band feature selection influences variance of θ power. To minimize the effect of alpha power variance on the real-time spectral estimations of θ power, the driving BCI feature was selected to be 4-6 Hz. Initial considerations were made based on prior evidence demonstrating the divergent frequency ranges of alpha amplitude, where the characteristic frequency ranges have previously been reported to be anywhere between 6 Hz and 13 Hz. Further, to mitigate the potential effect of blurring on the θ spectral estimation and the confounding impact of blurring from neighboring frequency bands (in this case alpha), a buffer area was established between the two frequency bands between 6 Hz and 8 Hz. The potentially confounding effect of including spectral power within this range was tested by calculating the variance of θ power change during the task-specific θ modulation screening. The variance of θ power change was calculated across all trials and subjects using either 4-6 Hz, 4-7 Hz, or 4-8 Hz as the frequency range for θ power. The variance in θ power change was found to be 0.079 μV2/Hz when using 4-6 Hz, 0.082 μV2/Hz when using 4-7 Hz, and 0.108 μV2/Hz when using 4-8 Hz.

A computer program of one embodiment is embodied on a computer-readable medium. In an example, the system is executed on a single computer system, without requiring a connection to a server computer. In a further example embodiment, the system is being run in a Windows® environment (Windows is a registered trademark of Microsoft Corporation, Redmond, Washington). In yet another embodiment, the system is run on a mainframe environment and a UNIX® server environment (UNIX is a registered trademark of X/Open Company Limited located in Reading, Berkshire, United Kingdom). In a further embodiment, the system is run on an iOS® environment (iOS is a registered trademark of Cisco Systems, Inc. located in San Jose, CA). In yet a further embodiment, the system is run on a Mac OS® environment (Mac OS is a registered trademark of Apple Inc. located in Cupertino, CA). In still yet a further embodiment, the system is run on Android® OS (Android is a registered trademark of Google, Inc. of Mountain View, CA). In another embodiment, the system is run on Linux® OS (Linux is a registered trademark of Linus Torvalds of Boston, MA). The application is flexible and designed to run in various different environments without compromising any major functionality. In some embodiments, the system includes multiple components distributed among a plurality of computing devices. One or more components are in the form of computer-executable instructions embodied in a computer-readable medium. The systems and processes are not limited to the specific embodiments described herein. In addition, components of each system and each process can be practiced independently and separately from other components and processes described herein. Each component and process can also be used in combination with other assembly packages and processes.

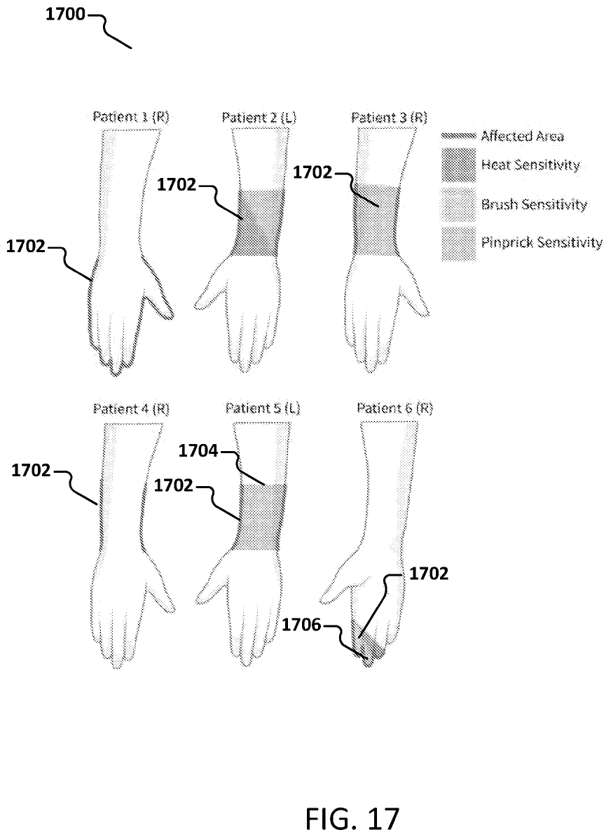

FIG. 17 shows a schematic of handedness, location, and hypersensitivity type of each patient's affected area. Heat sensitivity is shown as 1702. Brush sensitivity is shown as 1706. Pinprick sensitivity is shown as 1704.

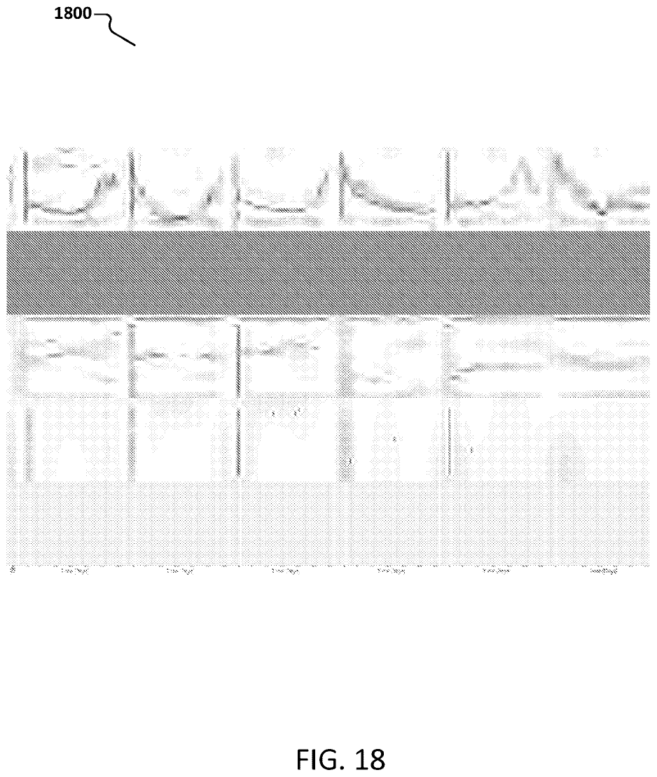

FIG. 18 shows power spectral density plots showing differences in frontal θ modulation during a serial subtraction mental arithmetic task (shading represents standard error). Comparison of frontal θ power during pre-BCI intervention baseline, and during BCI-neurofeedback (Wilcoxon rank sum, alpha=0.05). Normalized θ power across BCI sessions over time (Spearman's Correlation). θ modulation strength over time across BCI sessions. Total session time for each BCI training day. The solid line shows the overall trend by a line of best fit.

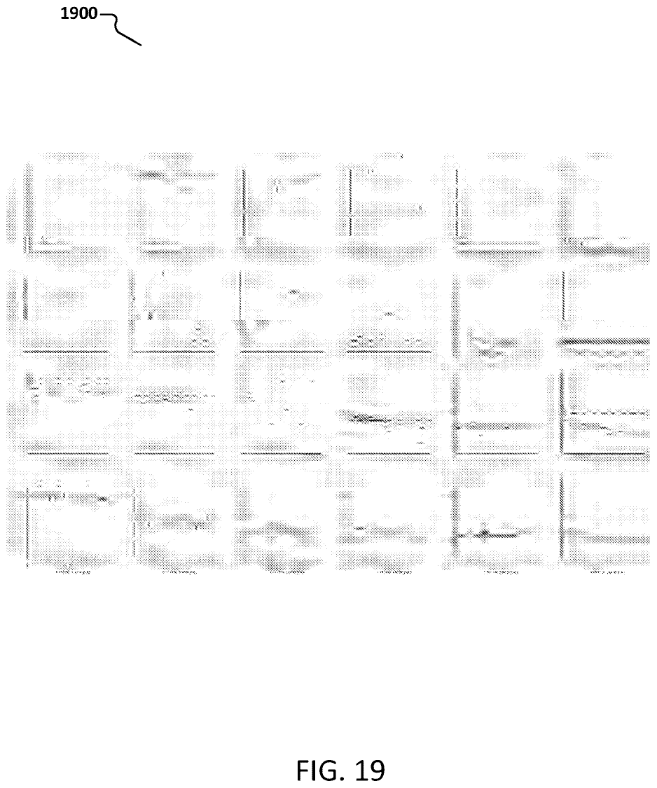

FIG. 19 shows plots of individual patient BCI performance and pain over BCI intervention. Each BCI session accuracy (completed trials/total trials) where the dotted line is accuracy during the first BCI day, median Fitts Metric of bit rate (bits/s) where the dashed line is the bit rate calculated for maximum duration of BCI control, pre-session VAS where the dashed line is the first pre-session VAS score, and post-session VAS where the dashed line is the first post-session VAS score on each day for each patient. Significant changes over time are shown by data points (Spearman correlation). The solid line shows the overall trend by a line of best fit.

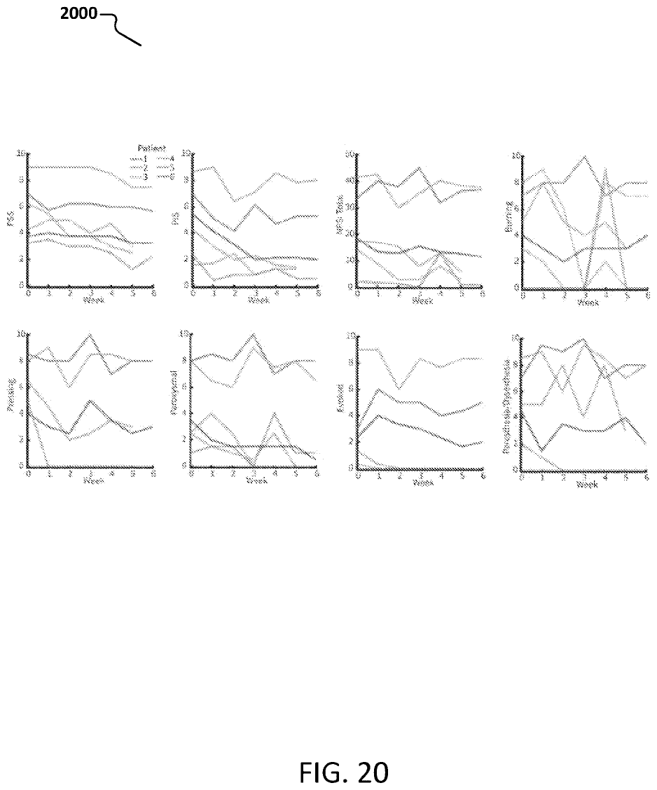

FIG. 20 shows plots of weekly BPI and NPSI scores over the intervention period for individual patients. That is, the plots show clinical outcomes (NPSI, PSS, and PIS) over BCI therapy for each patient. Measures were taken via a patient-reported survey given at the end of each week. The first three graphs show PSS, PIS, and NPSI. The last five graphs show symptom scores for NPSI.

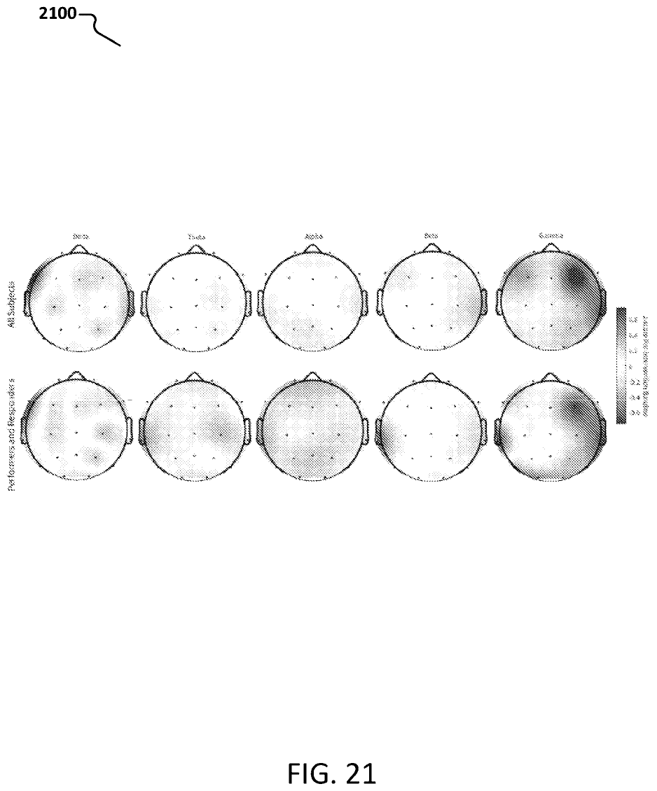

FIG. 21 shows topography plots of brain activity across patients before and after BCI intervention. The topography plots of baseline EEG activity are for all frequency bands after BCI therapy for all subjects (top) and for performers and VAS pain responders (bottom) z-scored to pre-intervention baseline.

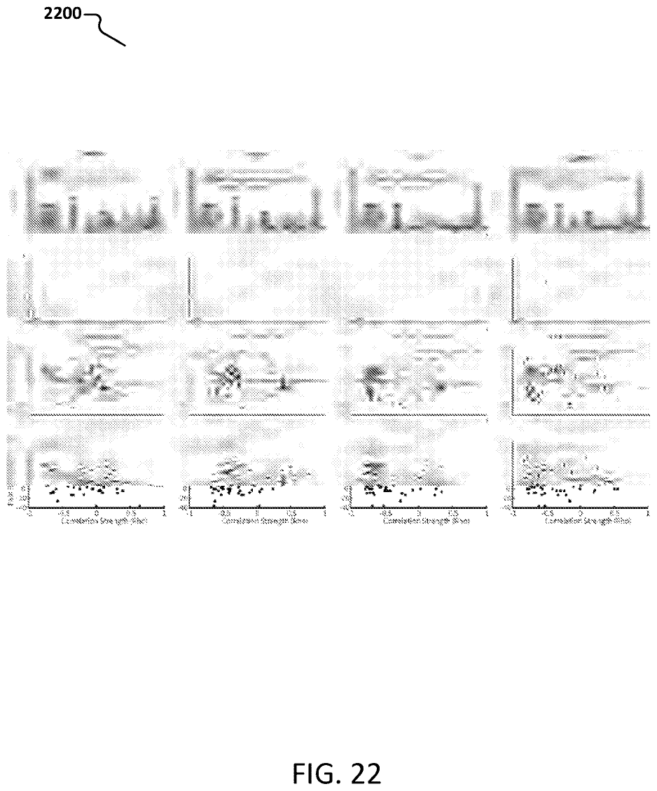

FIG. 22 shows correlation analysis of non-θ frequencies with pain relief. Secondary analysis of the relationship between (from left to right) delta, alpha, beta, and gamma modulation strength with BCI performance and pain relief. The first row shows a distribution of all BCI sessions across all patients with and without significant modulation of respective frequency bands. The dotted line represents the optimal classification threshold calculated by ROC analysis. The shaded bars represent sessions with significant power modulation relative to the pre-intervention baseline (Spearman correlation, p<0.05). The second row shows ROC curves demonstrating the true and false positive rates of correct classification of respective frequency modulation by performance value. The third row shows a relationship between respective frequency band modulation strength and pain relief using VAS obtained after each day's BCI session (Spearman correlation). A fourth row shows a relationship between respective frequency band modulation strength and pain relief using VAS obtained before each day's BCI session (Spearman correlation).

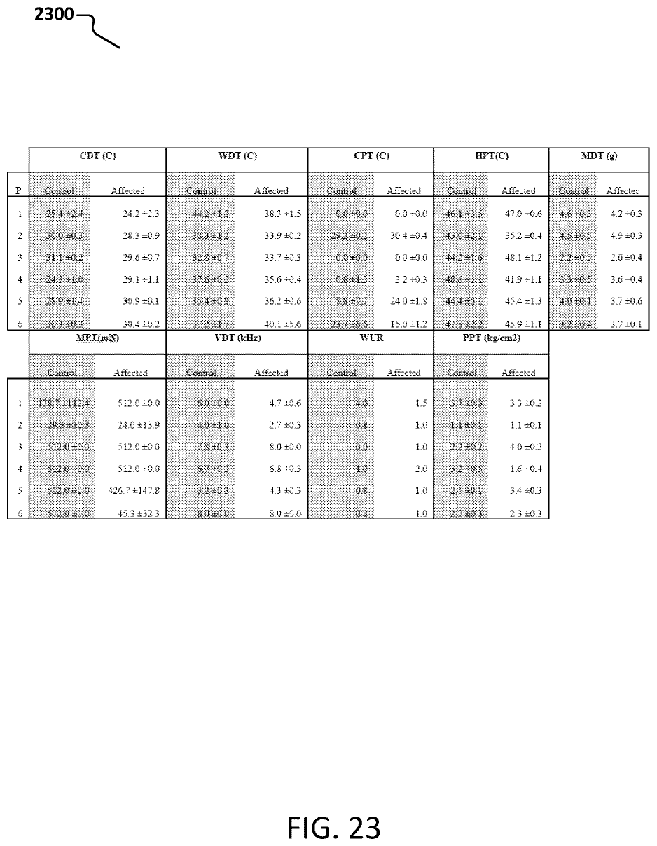

FIG. 23 includes a table showing patient quantitative sensory testing results. Quantitative sensory testing was used to determine differential sensory profiles of each participant's (P) affected (most painful) and unaffected (non-painful) regions. This was used to assess warm and cold detection (WDT and CDT) thresholds, heat and cold pain thresholds (HPT and CPT), mechanical detection and pain thresholds (MDT and MPT), presence of wind-up (enhanced temporal summation) to pinprick, vibration detection thresholds (VDT), and pressure pain threshold (PPT), and conditioned pain modulation (CPM). The Thermal Sensory Analyzer (TSA-II or PATHWAY platform; Medoc, Ramat Yishai, Israel) was used to determine WDT, CDT, HPT, and CPT. A set of standardized Semmes-Weinstein monofilaments (0.25, 0.5, 1, 2, 4, 8, 16, 32, 64, 128, and 256 mN; North Coast, USA) was used to assess MDT for each participant's affected and unaffected area. A set of calibrated blunt metal probes with applied force 8-512 mN (MRC systems, Germany) was used to determine the MPT. Wind-up ratio (WUR) was assessed using a #6.10 von Frey filament (980 mN) and calculated as the ratio between pain intensity elicited by a train of ten 1 Hz stimuli versus a single stimulus value was calculated. Each participant's frequency of vibration detection threshold was determined using a clinical tuning fork (64 Hz, 8/8 scale). A handheld algometer (Wagner Instruments) was used to determine each participant's PPT following continuous pressure application at a 0.5 kg/sec rate. The test sites used for affected and control sites were along the same muscle.

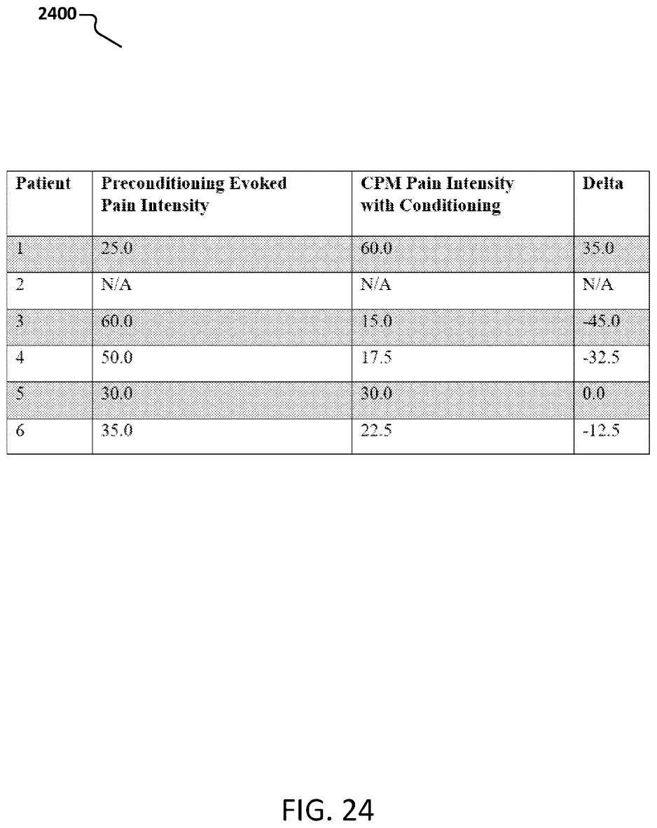

FIG. 24 is a table that includes patient conditioned pain modulation results. When tested for conditioned pain modulation (CPM) paradigm that assesses the efficiency of descending pain modulation, one patient had increased pain sensitivity during cold water conditioning, one patient had no change in pain sensitivity during cold water conditioning, and one patient could not tolerate the cold-water stimulus. CPM testing includes the application of a “test” stimulus without conditioning, and a subsequent application of the same test stimulus with conditioning. The test stimulus is generated via a thermode applied at a volar forearm, with temperature ramping at 1° C./sec to a temperature that elicits pain intensity of 60 on 0-100 NRS in the individual. The conditioning stimulus includes the immersion of the contralateral hand up to the wrist to a thermostat-controlled water bath maintained at 12□C. The length of the conditioning stimulus is 60 seconds, and during the last 30 seconds of it the test stimulus is applied (at the contralateral forearm) twice, as described above. The difference between the intensity of pain stimulus with concomitant conditioning and between the intensity of pain stimulus without conditioning is the CPM magnitude. CPM<0 implies efficient descending pain modulation.

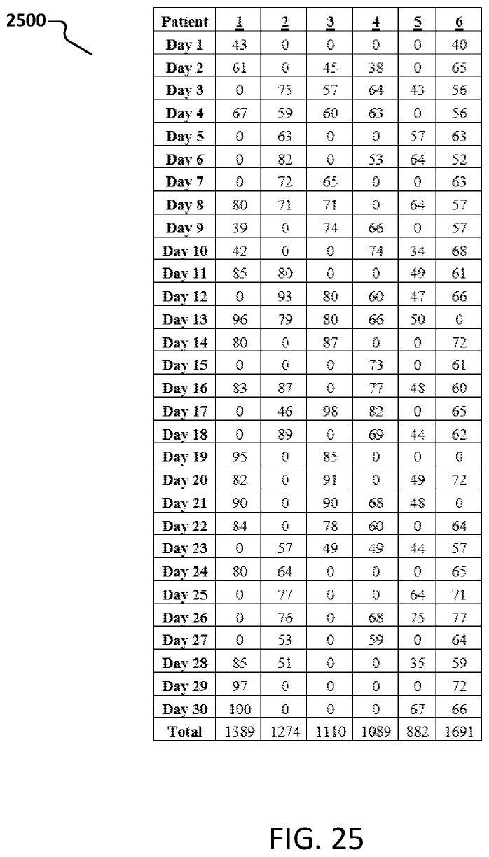

FIG. 25 is a table showing the number of BCI trials for each patient on each session day, and across the entire intervention period. Days with a “0” indicate days when the participant was not scheduled for BCI training during the 30-day intervention period.

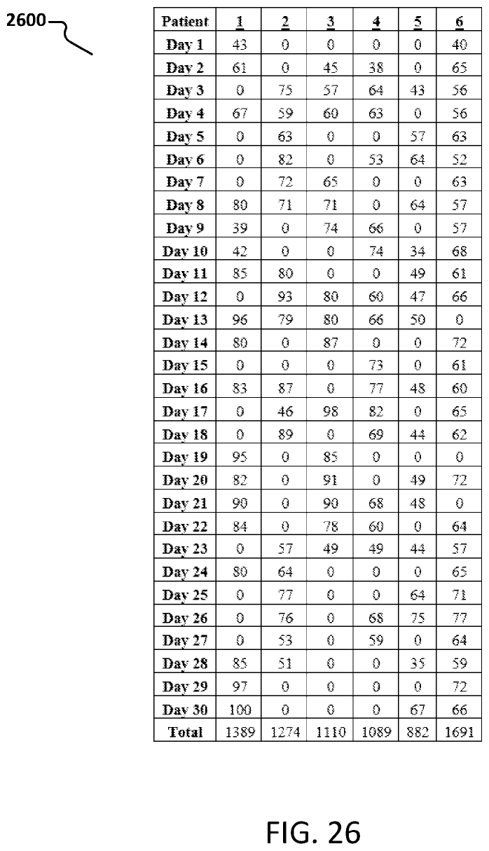

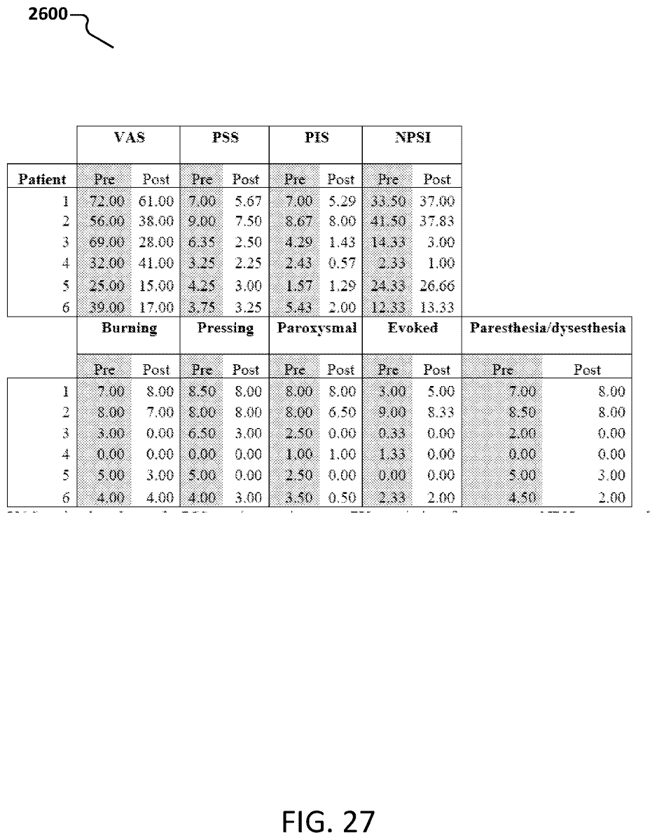

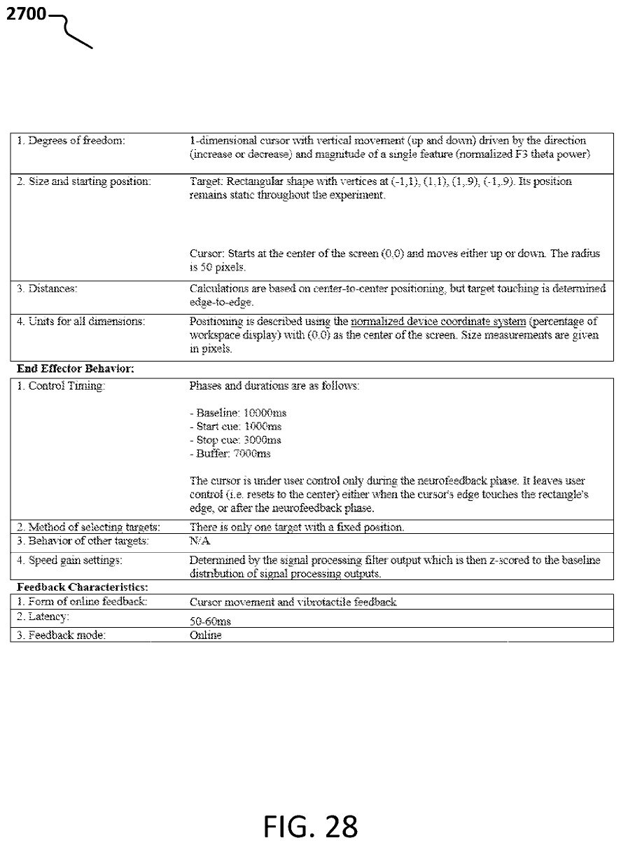

FIG. 26 shows a table that includes patient primary and secondary outcome measures. The bottom set contains scores for all sub-scores of NPSI. In the table: VAS=visual analog scale, PSS=pain severity score, PIS=pain interference score, NPSI=neuropathic pain symptom inventory score, Pre=pre-BCI intervention score, and Post=post-BCI intervention score. FIG. 27 is a table showing methods and features of the continuous BCI used in the study.

As used herein, the terms “processor” and “computer” and related terms, e.g., “processing device”, “computing device”, and “controller” are not limited to just those integrated circuits referred to in the art as a computer, but broadly refers to a microcontroller, a microcomputer, a programmable logic controller (PLC), an application specific integrated circuit (ASIC), and other programmable circuits, and these terms are used interchangeably herein. In the embodiments described herein, memory may include, but is not limited to, a computer-readable medium, such as a random-access memory (RAM), and a computer-readable non-volatile medium, such as flash memory. Alternatively, a floppy disk, a compact disc-read only memory (CD-ROM), a magneto-optical disk (MOD), and/or a digital versatile disc (DVD) may also be used. Also, in the embodiments described herein, additional input channels may be, but are not limited to, computer peripherals associated with an operator interface such as a mouse and a keyboard. Alternatively, other computer peripherals may also be used that may include, for example, but not be limited to, a scanner. Furthermore, in the exemplary embodiment, additional output channels may include, but not be limited to, an operator interface monitor.

Further, as used herein, the terms “software” and “firmware” are interchangeable and include any computer program storage in memory for execution by personal computers, workstations, clients, servers, and respective processing elements thereof.

As used herein, the term “non-transitory computer-readable media” is intended to be representative of any tangible computer-based device implemented in any method or technology for short-term and long-term storage of information, such as, computer-readable instructions, data structures, program modules and sub-modules, or other data in any device. Therefore, the methods described herein may be encoded as executable instructions embodied in a tangible, non-transitory, computer readable medium, including, without limitation, a storage device, and a memory device. Such instructions, when executed by a processor, cause the processor to perform at least a portion of the methods described herein. Moreover, as used herein, the term “non-transitory computer-readable media” includes all tangible, computer-readable media, including, without limitation, non-transitory computer storage devices, including, without limitation, volatile and nonvolatile media, and removable and non-removable media such as a firmware, physical and virtual storage, CD-ROMs, DVDs, and any other digital source such as a network or the Internet, as well as yet to be developed digital means, with the sole exception being a transitory, propagating signal.

Furthermore, as used herein, the term “real-time” refers to at least one of the time of occurrence of the associated events, the time of measurement and collection of predetermined data, the time for a computing device (e.g., a processor) to process the data, and the time of a system response to the events and the environment. In the embodiments described herein, these activities and events may be considered to occur substantially instantaneously.

The aspects described herein may be implemented as part of one or more computer components, such as a client device, system, and/or components thereof, for example. Furthermore, one or more of the aspects described herein may be implemented as part of a computer network architecture and/or a cognitive computing architecture that facilitates communications between various other devices and/or components. Thus, the aspects described herein address and solve issues of a technical nature that are necessarily rooted in computer technology.

A processor or a processing element may be trained using supervised or unsupervised machine learning, and the machine learning program may employ a neural network, which may be a convolutional neural network, a deep learning neural network, a reinforced or reinforcement learning module or program, or a combined learning module or program that learns in two or more fields or areas of interest. Machine learning may involve identifying and recognizing patterns in existing data in order to facilitate making predictions for subsequent data. Models may be created based upon example inputs in order to make valid and reliable predictions for novel inputs.

Additionally or alternatively, the machine learning programs may be trained by inputting sample data sets or certain data into the programs, such as images, object statistics and information, traffic timing, previous trips, and/or actual timing. The machine learning programs may utilize deep learning algorithms that may be primarily focused on pattern recognition and may be trained after processing multiple examples. The machine learning programs may include Bayesian Program Learning (BPL), voice recognition and synthesis, image or object recognition, signal processing, optical character recognition, and/or natural language processing-either individually or in combination. The machine learning programs may also include natural language processing, semantic analysis, automatic reasoning, and/or machine learning.

Supervised and unsupervised machine learning techniques may be used. In supervised machine learning, a processing element may be provided with example inputs and their associated outputs and may seek to discover a general rule that maps inputs to outputs, so that when subsequent novel inputs are provided the processing element may, based upon the discovered rule, accurately predict the correct output. In unsupervised machine learning, the processing element may be required to find its own structure in unlabeled example inputs. In one embodiment, machine learning techniques may be used to determine brain responses to stimuli such as settings of the vibrotactile device and/or brainwave activations that lead to the most relief from pain in patients.

Based upon these analyses, the processing element may learn how to identify characteristics and patterns that may then be applied to analyzing image data, model data, and/or other data. For example, the processing element may learn, to identify brain responses to stimuli and the best brainwave activations for relief for different patients to provide optimal brainwave activity and stimulus. The processing element may also learn how to identify trends that may not be readily apparent based upon collected data, such as trends that identify when brainwave activity will spike or decline.

The exemplary systems and methods described and illustrated herein therefore provide a non-invasive electroencephalography (EEG)-based BCI coupled to a wearable exoskeleton (e.g., the vibrotactile device) that is capable of remodeling neural circuits in the setting of chronic pain and significantly improving patient quality of life.

The computer-implemented methods and processes described herein may include additional, fewer, or alternate actions, including those discussed elsewhere herein. The present systems and methods may be implemented using one or more local or remote processors, transceivers, and/or sensors (such as processors, transceivers, and/or sensors mounted on vehicles, stations, nodes, or mobile devices, or associated with smart infrastructures and/or remote servers), and/or through implementation of computer-executable instructions stored on non-transitory computer-readable media or medium. Unless described herein to the contrary, the various steps of the several processes may be performed in a different order, or simultaneously in some instances.

Additionally, the computer systems discussed herein may include additional, fewer, or alternative elements and respective functionalities, including those discussed elsewhere herein, which themselves may include or be implemented according to computer-executable instructions stored on non-transitory computer-readable media or medium.

In the exemplary embodiment, a processing element may be instructed to execute one or more of the processes and subprocesses described above by providing the processing element with computer-executable instructions to perform such steps/sub-steps, and store collected data (e.g., trust stores, authentication information, etc.) in a memory or storage associated therewith. This stored information may be used by the respective processing elements to make the determinations necessary to perform other relevant processing steps, as described above.

The aspects described herein may be implemented as part of one or more computer components, such as a client device, system, and/or components thereof, for example. Furthermore, one or more of the aspects described herein may be implemented as part of a computer network architecture and/or a cognitive computing architecture that facilitates communications between various other devices and/or components. Thus, the aspects described herein address and solve issues of a technical nature that are necessarily rooted in computer technology.

Although specific features of various embodiments may be shown in some drawings and not in others, this is for convenience only. In accordance with the principles of the systems and methods described herein, any feature of a drawing may be referenced or claimed in combination with any feature of any other drawing.

Some embodiments involve the use of one or more electronic or computing devices. Such devices typically include a processor, processing device, or controller, such as a general purpose central processing unit (CPU), a graphics processing unit (GPU), a microcontroller, a reduced instruction set computer (RISC) processor, an application specific integrated circuit (ASIC), a programmable logic circuit (PLC), a programmable logic unit (PLU), a field programmable gate array (FPGA), a digital signal processing (DSP) device, and/or any other circuit or processing device capable of executing the functions described herein. The methods described herein may be encoded as executable instructions embodied in a computer readable medium, including, without limitation, a storage device and/or a memory device. Such instructions, when executed by a processing device, cause the processing device to perform at least a portion of the methods described herein. The above examples are exemplary only, and thus are not intended to limit in any way the definition and/or meaning of the term processor and processing device.

The computer-implemented methods discussed herein may include additional, less, or alternate actions, including those discussed elsewhere herein. The methods may be implemented via one or more local or remote processors, transceivers, servers, and/or sensors, and/or via computer-executable instructions stored on non-transitory computer-readable media or medium.

Additionally, the computer systems discussed herein may include additional, less, or alternate functionality, including that discussed elsewhere herein. The computer systems discussed herein may include or be implemented via computer-executable instructions stored on non-transitory computer-readable media or medium.

This written description uses examples to describe the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A system for treating chronic pain in a patient, the system comprising:
a user computing device including a display screen; and
a brain-computer interface (BCI) device communicatively coupled to the user computing device and comprising a processor communicatively coupled to an electroencephalogram (EEG) headset and a vibrotactile device, wherein the EEG headset is configured to be attached to the patient and to monitor brainwaves of the patient, wherein the vibrotactile device is configured to be coupled to an anatomic region of the patient and generate multi-modal sensory feedback, and wherein the processor is configured to:
prompt, through the display screen, the patient to carry out a plurality of cognitive tasks, the plurality of cognitive tasks selected to create theta activation brainwaves in the patient;
monitor, via feedback from the EEG headset, progress of the patient toward reaching a predetermined theta activation goal;
display, through the display screen, a visual feedback via a cue showing the progress of the patient in relation to the predetermined theta activation goal; and
activate, in response to the patient reaching the predetermined theta activation goal, the vibrotactile device to generate the multi-modal sensory feedback.

2. The system of claim 1, wherein the vibrotactile device comprises:
control and driving circuits;
an adjustable frame configured to accommodate a range of anatomic regions of the patient, wherein the adjustable frame is configured to ratchet closed for secure vibration; and
a set of repositionable and interchangeable components configured to be removably coupled to the adjustable frame.

3. The system of claim 2, wherein the set of repositionable and interchangeable components comprise:
a plurality of nodes configured to (i) deliver vibration to skin of the patient and (ii) dampen the vibration from entering the adjustable frame; and
a plurality of buffers configured to (i) further isolate the vibration and (ii) provide support at body contact points for effective fixation onto the anatomic region of the patient.

4. The system of claim 1, wherein the processor is further configured to:
statistically compare, in real-time or near real-time, current task performance and baseline data to monitor the progress of the patient.

5. The system of claim 4, wherein the baseline data includes a frequency representative of a theta frequency identifier, and wherein the frequency ranges in between 4-7 Hz.

6. The system of claim 1, wherein the processor is further configured to:
identify individual brain rhythms for the patient associated with pain relief that are optimal for patient control.

7. The system of claim 1, wherein the EEG headset is a dry electrode EEG headset.

8. A method for treating chronic pain in a patient, the method comprising:
prompting, through a display screen of a user computing device, a patient to carry out a plurality of cognitive tasks, the plurality of cognitive tasks selected to create theta activation brainwaves in the patient;
monitoring, via feedback from an electroencephalogram (EEG) headset, progress of the patient toward reaching a predetermined theta activation goal, wherein the EEG headset is configured to be attached to the patient and to monitor brainwaves of the patient, wherein the EEG headset and the user computer device are coupled to a brain-computer interface (BCI) device;
displaying, through the display screen, a visual feedback via a cue showing the progress of the patient in relation to the predetermined theta activation goal; and
activating, in response to the patient reaching the predetermined theta activation goal, generating multi-modal sensory feedback, wherein the vibrotactile device is configured to be coupled to an anatomic region of the patient and to the generate the multi-modal sensory feedback, and wherein the vibrotactile device is coupled to the BCI device.

9. The method of claim 8, further comprising configuring the vibrotactile device to include an adjustable frame to accommodate a range of anatomic regions of the patient, wherein the adjustable frame is configured to ratchet closed for secure vibration using a set of repositionable and interchangeable components configured to be removably coupled to the adjustable frame.

10. The method of claim 9, further comprising delivering vibration to skin of the patient and dampening the vibration from entering the adjustable frame using a plurality of nodes of the vibrotactile device.

11. The method of claim 8, further comprising isolating vibration and providing support at body contact points for effective fixation onto the anatomic region of the patient using a plurality of buffers.

12. The method of claim 8, further comprising statistically comparing, in real-time or near real-time, current task performance and baseline data to monitor the progress of the patient.

13. The method of claim 12, further comprising setting the baseline data to include a frequency representative of a theta frequency identifier, wherein the frequency ranges in between 4-7 Hz.

14. The method of claim 8, further comprising identifying individual brain rhythms for the patient associated with pain relief that are optimal for patient control.

15. At least one non-transitory computer-readable storage medium with instructions stored thereon that, in response to execution by at least one processor, cause the at least one processor to:
prompt, through a display screen of a user computing device, a patient to carry out a plurality of cognitive tasks, the plurality of cognitive tasks selected to create theta activation brainwaves in the patient;
monitor, via feedback from an electroencephalogram (EEG) headset, progress of the patient toward reaching a predetermined theta activation goal, wherein the EEG headset is configured to be attached to the patient and to monitor brainwaves of the patient, wherein the EEG headset and the user computer device are coupled to a brain-computer interface (BCI) device;
display, through the display screen, a visual feedback via a cue showing the progress of the patient in relation to the predetermined theta activation goal; and
activate, in response to the patient reaching the predetermined theta activation goal, a vibrotactile device to generate multi-modal sensory feedback, wherein the vibrotactile device is configured to be coupled to an anatomic region of the patient, and wherein the vibrotactile device is coupled to the BCI device.

16. The at least one non-transitory computer-readable storage medium of claim 15, wherein the at least one processor is further configured to initiate a delivery of vibration to skin of the patient and to initiate dampening the vibration.

17. The at least one non-transitory computer-readable storage medium of claim 15, wherein the at least one processor is further configured to statistically compare, in real-time or near real-time, current task performance and baseline data to monitor the progress of the patient.

18. The at least one non-transitory computer-readable storage medium of claim 17, wherein the at least one processor is further configured to initiate setting the baseline data to include a frequency representative of a theta frequency identifier

19. The at least one non-transitory computer-readable storage medium of claim 18, wherein the frequency is about 5 Hz.

20. The at least one non-transitory computer-readable storage medium of claim 15, wherein the at least one processor is further configured to identify individual brain rhythms for the patient associated with pain relief that are optimal for patient control.
